SEQUENCE LISTING (4 sequences)

SEQ ID NO: 1 - protein, 506 aa, Homo sapiens
MASGDLYEVERIVDKRKNKKGKWEYLIRWKGYGSTEDTWEPEHHLLHCEEFIDEFNGLHMSKDKRIKSGKQSSTSKLLRDSRGPSVEKLSHRPSDPGKSKGTSHKRKRINPPLAKPKKGYSGKPSSGGDRATKTVSYRTTPSGLQIMPLKKSQNGMENGDAGSEKDERHFGNGSHQPGLDLNDHVGEQDMGECDVNHATLAENGLGSALTNGGLNLHSPVKRKLEAEKDYVFDKRLRYSVRQNESNCRFRDIVVRKEEGFTHILLSSQTSDNNALTPEIMKEVRRALCNAATDDSKLLLLSAVGSVFCSGLDYSYLIGRLSSDRRKESTRIAEAIRDFVKAFIQFKKPIVVAINGPALGLGASILPLCDIVWASEKAWFQTPYATIRLTPAGCSSYTFPQILGVALANEMLFCGRKLTAQEACSRGLVSQVFWPTTFSQEVMLRVKEMASCSAVVLEESKCLVRSFLKSVLEDVNEKECLMLKQLWSSSKGLDSLFSYLQDKIYEV

SEQ ID NO: 2 - DNA, 1521 bp, Homo sapiens
atggcttctggggacctttacgaggttgaaaggattgtagacaagaggaagaacaagaaaggaaaatgggagtatcttatccgatggaaaggctacgggagcaccgaggacacgtgggagccggagcaccacctcttgcactgtgaggagtttattgatgaattcaatgggttgcacatgtccaaggacaagaggatcaagtcagggaagcagtccagtacctccaagctgctgcgtgacagtcgaggcccgtcggttgagaaactgtcccacagaccttcagatcctggaaagagcaaggggacctcccataaacggaagcgaattaaccctcccctggccaagccaaaaaaagggtattcaggcaagccctcttcaggaggtgacagggccaccaagacggtgtcttacaggactacccccagtggtttgcaaataatgcccctgaaaaagtctcagaacgggatggaaaatggggacgccggctctgagaaggatgagaggcactttggaaatgggtcccatcagcctggcttggatttgaatgatcatgttggagagcaagatatgggtgaatgtgacgtgaatcacgctacactggcggagaacgggctcggctctgctctgaccaacgggggattgaacctgcacagtccagtgaagaggaagctggaagcggagaaggactacgtctttgacaaaaggctcagatacagtgtccgccagaatgaaagcaactgtcggtttcgagacatcgttgtgcggaaggaagaagggttcacgcacatcctgctgtccagtcagacctcggataacaatgccctgacacctgagatcatgaaagaagtccggcgagcgctctgcaacgcagccacagacgacagcaaactgctgctcctcagcgcagtggggagcgtgttctgcagcggcctggattattcctacctaattggccggttgtccagcgaccggcgaaaggagagcactcggattgcagaagccatcagggactttgtgaaggcctttatccagtttaagaagcctatcgtggtggccatcaatgggccggccctgggcctgggtgcctccatcctgcccctctgtgacatcgtgtgggccagtgagaaggcctggttccagacgccctacgccaccatccgcctcacgcctgctggctgctcctcctacaccttcccccagatcctgggcgtcgcgctggccaatgagatgctgttctgtgggcggaagctcaccgcccaggaggcctgcagcagggggctggtgtcgcaggtcttctggcccaccacgttcagccaggaggtcatgctgcgggtcaaggagatggcatcctgcagtgccgtggtgttagaggagtccaaatgcctcgtgcggagcttcctgaaatcagtgctggaagacgtgaacgagaaggaatgcctcatgctcaagcagctctggagctcctccaaaggccttgactcccttttcagctacctgcaggacaaaatttatgaagtctga

SEQ ID NO: 3 - DNA, 22 bp, Artificial
  Synthetic CDYL2 forward PCR primer
accaacgggggattgaacctgc

SEQ ID NO: 4 - DNA, 25 bp, Artificial
  Synthetic CDYL2 PCR reverse primer
ggtgtcagggcattgttatccgagg

(19) United States
(12) Patent Application Publication
(10) Pub. No.: US 2023/0070181 A1
(43) Pub. Date: Mar. 9, 2023

(54) METHODS OF TREATMENT OF CANCER DISEASE BY TARGETING AN EPIGENETIC FACTOR

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); UNIVERSITÉ CLAUDE BERNARD - LYON 1, Villeurbanne (FR); CENTRE LEON BERARD, Lyon (FR)

(72) Inventors: Peter MULLIGAN, Lyon - Cedex 08 (FR); Pierre SAINTIGNY, Lyon - Cedex 08 (FR); Maha SIOUDA, Lyon (FR)

(21) Appl. No.: 17/760,024

(22) PCT Filed: Feb. 4, 2021
(86) PCT No.: PCT/EP2021/052593

§ 371 Date: Aug. 3, 2022

(30) Foreign Application Priority Data
Feb. 5, 2020 (EP) 20305101.6

(51) Int. Cl. A61K 31/7105; A61K 31/713; C07K 16/30; C07K 16/32; A61P 35/00

(52) U.S. Cl. CPC A61K 31/7105; A61K 31/713; C07K 16/3015; C07K 16/32; A61P 35/00

(57) ABSTRACT

The present invention relates to a method for preventing or treating a cancer disease by targeting the epigenetic factor Chromodomain on Y-like 2 (CDYL2). The inventors found that CDYL2 is commonly over-expressed in cancer and high CDYL2 levels correlate with poor prognosis in a number of cancer types even in drug resistant cancer. CDYL2 upregulation in a breast cancer cell line induced migration, invasion, stem-like phenotypes, as well as an epithelial-to-mesenchymal transition (EMT). Due to the importance of EMT and stemness in therapeutic resistance and relapse in cancer, the inventors propose that CDYL2 inhibition will also be beneficial to the treatment of such cancers. Furthermore RNAi inhibition of CDYL2 diminished these same EMT-associated processes in the mesenchymal-like breast cancer cell line. Finally ablating the expression of CDYL2 with RNAi 1) stimulates the expression of genes associated with an anti-tumor immune response (such as gene involved in interferon response) and 2) inhibits lung tumorigenesis in a preclinical model (mouse injected with the triple negative MDA-MB-231 cell line). These results show that CDYL2 as a strong candidate proto-oncogene and therapeutic target in cancer and also contributes to the anti-tumoral immune response escape.

### FIELD OF THE INVENTION

The present invention relates to a method for preventing or treating a cancer disease by targeting the epigenetic factor Chromodomain on Y-like 2 (CDYL2). The invention also relates to a CDYL2 antagonist for use (i) in a method to activate the anti-tumoral immune response of a patient affected with a cancer and (ii) for use in the prevention or treatment of a patient affected with a cancer resistant disease.

### BACKGROUND OF THE INVENTION

Epigenetic factors that modify and bind histones orchestrate gene expression programs in a manner that is durable, yet can be altered in a controlled manner. In this way, they regulate transitions in cell identity and function underlying development and cellular differentiation. Epigenetic perturbations are prevalent in cancer and can cause cell identity transitions favoring malignant progression, including epithelial-to-mesenchymal transitions (EMT) and the acquisition of stem-like properties^{1,2}. Inhibition of epigenetic factors that promote such cancer cell plasticity might offer new opportunities for cancer treatment. However, the molecular links between epigenetic perturbations and malignancy-associated plasticity remain poorly understood, and several putative epigenetic factors remain uncharacterized. Gene expression changes underlying EMT and stemness result from interconnected regulatory systems involving transcription factors, epigenetic factors and non-coding RNAs. In breast cancer, active forms of the transcription factors p65/RelA and STAT3 promote EMT, migration, invasion and stemness³⁻⁸. Misregulation of the histone methyltransferases EZH2 and G9a can also induce these cellular processes⁹⁻¹². An important role has emerged for the microRNA-124 (miR-124), which targets numerous mRNAs to suppress cancer cell invasion, stemness and EMT, and is often silenced in breast cancer¹³⁻¹⁵ Critical roles have emerged for STAT3 and NF-κB inhibition in the mechanism of miR-124 action¹⁵⁻²⁰. Recently, EZH2 was implicated in miR-124 repression in renal carcinoma cells²¹, supporting a role for histone modifications in MIR124 silencing in cancer. Notably, molecular and cellular changes associated with EMT and stemness in cancer cells were proposed to underlie resistance to a range of cancer therapies, as well as increased propensity to form invasive and metastatic tumors (^{42-44; 57, 59}).

In this study, we investigated the molecular and cellular functions of the putative epigenetic factor Chromodomain on Y-like 2 (CDYL2) in breast cancer. This is a member of the CDYL family of genes, which includes two autosomal homologs in humans, CDYL1/CDYL, and CDYL2²². The family is defined by the presence of an N-terminal chromodomain that binds to methylated histone H3 lysine 9 (H3K9) and H3K27 residues^{23,24}, and a C-terminal domain homologous to enoyl coenzyme A hydratase/isomerase enzymes²². CDYL1 is implicated in cancer as a candidate oncogene or tumor suppressor, depending on the context^{25,26}, and its epigenetic mechanism involves its interaction with and regulation of several other epigenetic factors, notably the H3K9 methyltransferases G9a/EHMT2, GLP/EHMT1 and SETDB1/ESET²⁵, and EZH2²⁷. By contrast, very little is known about the roles of CDYL2 in physiology or disease or its putative epigenetic mechanism.

A potential role of CDYL2 in cancer was suggested by a Genome-wide Association Study (GWAS) that identified an intronic single nucleotide polymorphism (SNP) in CDYL2 associated with cancer risk²⁸. Here inventors show that CDYL2 expression is also frequently upregulated in breast cancer, and that high expression correlates with poor outcome in the estrogen receptor-positive/human epidermal growth factor receptor 2-negative (ER+/HER2−) and triple negative (TN) subtypes. Inventors propose that high levels of CDYL2 expression promote epigenetic repression of MIR124 genes by increasing G9a and EZH2 recruitment and H3K9 and H3K27 methylation at upstream regulatory regions. This, in turn, contributes to CDYL2 induction of NF-1B and STAT3 signaling, consequent induction of EMT genes, and increased cell motility, invasiveness, and stemness. These findings implicate CDYL2 as candidate proto-oncogene and therapeutic target in cancer (in solid tumor such as in breast, colorectal, esophagus cancers and also leukemia).

The purpose of the present invention is therefore to address this need by providing: a new therapeutic target for treating cancer and cancer drug resistant disease.

Cancer cells express gene products that produce neoantigens for presentation on class I MHC receptors on tumor cells, or class I and class II MHC receptors on other antigen presenting cell types within the tumor microenvironment. Cancer neoantigens can be derived from the transcription of genomic regions not normally expressed, for instance the Cancer/Testis Antigen (CTA) genes or the expression of mutated genes. In addition, in the case of virus-associated cancers, viral gene products can give rise to non-self antigens for presentation on class I and class II MHC receptors. When recognized by T cells, these MHC-peptide complexes have the potential to induce T cell activation, and thus T cell-directed elimination of the tumor cell. However, during the process of tumorigenesis, cancer cells avoid such detection and elimination by the immune system in a process termed immune evasion (Bobisse et al., (2016) Ann Transl Med; Topper et al., (2019) Nat Rev Clin Oncol).

Immune evasion can be achieved by interrupting the anti-tumor immune response at various critical points. These include: 1) the infiltration of T cells into the tumor microenvironment; 2) the presentation of neoantigens, or viral antigens, in complexes with MHC receptors; 3) the activation of T cells, via expression of co-stimulatory ligands and the absence of expression of immune checkpoint ligands. Due to the success of immune checkpoint inhibitors (ICIs) in treating certain cancer types, notably melanoma and non-small cell lung cancer (NSCLC), identifying ways to expand the use of ICIs to a broader range of cancer types is an important therapeutic goal. It remains unclear why certain cancers do not respond to ICIs. Among the best supported hypotheses are that they do not have sufficient T cell infiltrates in the tumor microenvironment (‘immune cold’ tumors), or do not present neoantigens or viral antigens at sufficiently high levels for T cell activation due to down-regulation of the antigen processing and presentation machinery (immunoediting). Therefore, therapies that induce T cell infiltration or up-regulate tumor antigen presentation could induce an anti-tumor immune response either alone, or in conjunction with ICI treatment. Among the most promising of such strategies to have emerged involves combining inhibitors of DNA- and histone-modifying enzymes with ICIs. Specifically, it was observed that inhibitors of DNA- and histone modifying enzymes that resulted in up-regulation of the interferon (IFN) response in tumor cells promoted sensitivity of those tumors to ICIs. However, this therapeutic approach remains at the experimental stage, and so far has focused largely on epigenetic drugs that are FDA approved for cancer treatment in other contexts, namely DNA methyltransferase inhibitors (DNMTi) and Histone deacetylase inhibitors (HDACi) (Bobisse et al., (2016) Ann Transl Med; Topper et al., (2019) Nat Rev Clin Oncol).

The ability of DNMTi and HDACi to induce an interferon (IFN) response in tumor cells appears to be fundamental to the priming of such tumors to respond to ICI treatment. Activation of the IFN response in tumor cells elicits a broadly pro-inflammatory effect. Notable aspects include upregulation of the antigen-presentation apparatus, resulting in increased capacity to present tumor antigens to T cells, and secretion of cytokines that attract T cells to the tumor microenvironment. However, in many tumor contexts, the IFN response also increases expression of the immune checkpoint activating ligand PD-L1, high levels of which block T cell activation. Hence, even if the tumor cells present more non-self antigens and attract more T cells (so called ‘immune hot’ tumors), they do not elicit an anti-tumor immune response. However, they appear to be primed to respond to ICIs. Treatments that elicit an IFN response in tumor cells could increase the number of cancer patients that can benefit from the remarkable efficacy of immune checkpoint inhibitors (Minn and Wherry, (2016) Cell; Topper et al., (2019) Nat Rev Clin Oncol).

The molecular basis of epigenetic control of the cell-intrinsic IFN response in tumor cells remains incompletely understood. Several studies reported that epigenetic silencing of DNA sequences encoding non-coding RNA (ncRNA) species such as endogenous retroviruses (ERVs) and satellite DNA repeats play a crucial role. Elevated expression of certain of these ncRNAs, notably ncRNA that can form double-stranded RNAs (dsRNA), was shown to activate the intracellular viral dsRNA response, which in turn elicits the IFN response pathway. Treatment of tumor cell lines with inhibitors of DNMT, HDAC, Lysine-specific demethylase 1 (LSD1) and RNA interference (RNAi) knock-down or genetic interruption of SETDB1 all resulted in up-regulation of ERV and other dsRNA species in tumor cells of various origin, accompanied by an IFN response. The mechanism of up-regulation of these dsRNA upon DNA methylation, HDAC, LSD1 and SETDB1 inhibition or loss was proposed to involve deregulation of DNA cytosine methylation and/or histone modifications at the dsRNA-encoding loci (Chiappinelli et al., (2015) Cell; Cuellar et al., (2017) J Cell Biol; Sheng et al., (2018) Cell). However, additional mechanisms were also proposed, such as LSD1 inhibitor-induced loss of demethylation of the RNA-induced silencing complex (RISC), resulting in diminished RISC activity and dsRNA accumulation (Sheng et al., 2018). Clinical trials are already underway to assess the benefit of co-treating cancer patients with both ICI and FDA-approved DNMT and/or HDAC inhibitors (Topper et al., (2019) Nat Rev Clin Oncol). Studies from inventors showed that the putative epigenetic regulator Chromodomain on Y-like 2 (CDYL2) is frequently up-regulated in breast cancer and correlates with poor prognosis. Here inventors present evidence that RNAi knock-down of CDYL2 induces an IFN response in the human breast cancer cell lines MCF-7 and MDA-MB-231. Accordingly the present invention proposes that therapeutic inhibition of CDYL2 could also be used as a means to increase the anti-cancer immune response.

### SUMMARY OF THE INVENTION

A first object of the invention relates to CDYL2 antagonist for use in the prevention or treatment of a patient affected with a cancer disease.

In a particular embodiment, the cancer is a drug resistant cancer.

A second object of the invention relates to a CDYL2 antagonist for use in a method to activate the anti-tumoral immune response of a patient affected with a cancer.

### DETAILED DESCRIPTION OF THE INVENTION

Here the inventors investigated the correlation between CDYL2 biopsy levels and biological findings from cancer patients. They found that surprisingly: 1) CDYL2 is commonly over-expressed in breast cancer, and high CDYL2 levels correlate with poor prognosis in the ER+/HER2- and triple negative (TN) sub-types of breast resistant cancer (FIG. 1). High CDYL2 levels also correlate with poor prognosis in colorectal, rectal and lung cancers (FIG. 8) 2) CDYL2 upregulation in the epithelioid breast cancer cell line MCF7 induced migration, invasion, stem-like phenotypes, as well as an apparent epithelial-to-mesenchymal transition (EMT) (FIG. 3) 3) RNAi inhibition of CDYL2 diminished these same plasticity-associated processes in the mesenchymal-like breast cancer cell line MDA-MB-231 (see FIG. 4) 4) CDYL2 induction of EMT genes, invasion and stemness in MCF7 cells depended on signaling via p65/NF-κB and STAT3 (FIGS. 5 and 6) 5) Co-immunoprecipitation studies revealed that CDYL2 formed a complex with, and regulated the chromatin enrichment of, histone methyltransferase G9a and histone H3 lysine 9 dimethylation (H3K9me2) upstream of MIR124 family genes and also regulated the enrichment of the polycomb group methyltransferase EZH2 and H3K27 trimethylation (H3K27me3) upstream of MIR124 genes (FIG. 7) 6) ablating the expression of CDYL2 with RNAi stimulates anti-tumor immunity by inducing IFN-response genes (see Example 2, Table 1 to 7)) 7) ablating the expression of CDYL2 with RNAi inhibits lung tumorigenesis in a preclinical model (mouse injected with the triple negative MDA-MB-231 cell line) (see Example 3, Table 9, FIG. 10). These results show that CDYL2 as a strong candidate proto-oncogene and therapeutic target in cancer and also contributes to the anti-tumoral immune response blockade. Accordingly Neutralizing CDYL2, which acts as a proto-oncogene controlling cancer cell migration, invasion, stemness and plasticity and the expression of genes involved in regulating the anti-tumor immune response (such as genes involved in IFN response) in cancer, therefore could allow to block tumor transition and furthermore restore beneficial anti-tumor immunity in cancer.

### Therapeutic Methods and Uses

The present invention provides methods and compositions (such as pharmaceutical compositions) for preventing or treating a cancer disease. The present invention also provides methods and compositions for inhibiting or preventing cancer disease.

In the context of the invention, the term “treatment or prevention” means reversing, alleviating, inhibiting the progress of, or preventing the disorder or condition to which such term applies, or one or more symptoms of such disorder or condition. In particular, the treatment of the disorder may consist in reducing the number of malignant cells. Most preferably, such treatment leads to the complete depletion of the malignant cells.

Preferably, the individual to be treated is a human or non-human mammal (such as a rodent (mouse, rat), a feline, a canine, or a primate) affected or likely to be affected with cancer.

Preferably, the individual is a human.

According to a first aspect, the present invention relates to a CDYL2 antagonist for use in the prevention or the treatment of a patient affected with a cancer disease.

As used herein the term “CDYL2” (Chromodomain on Y-like 2) also known as “PCCP1”, has its general meaning in the art. CDYL2 is a member of the CDYL family of genes, which includes two autosomal homologs in humans, CDYL1/CDYL, and CDYL2²². The CDYL family is defined by the presence of an N-terminal chromodomain that binds to methylated histone H3 lysine 9 (H3K9) and H3K27 residues^{23,24}, and a C-terminal domain homologous to enoyl coenzyme A hydratase/isomerase enzymes²². One example of wild-type CDYL2 human amino acid sequence is provided in SEQ ID NO:1 (UniProtKB—Q8N8U2/NCBI Reference Sequence: NP_689555) (table 8). One example of nucleotide sequence encoding wild-type CDYL2 is provided in SEQ ID NO:2 (NCBI Reference Sequence: NM_152342) (table 8).

Of course variant sequences of the CDYL2 may be used in the context of the present invention, those including but not limited to functional homologues, paralogues or orthologues of such sequences such as:

CDYL2 isoform X1: (NCBI Reference Sequence: XM_011522866.1/XP_011521168.1/GI: 767989391)

CDYL2 isoform X2: (NCBI Reference Sequence: XM_011522867.2/XP_011521169.1/GI: 1034593618)

CDYL2 isoform X3: (NCBI Reference Sequence: XM_024450151.1/XP_024305919.1/GI: 1370467935)

A “CDYL2 antagonist” refers to a molecule (natural or synthetic) capable of neutralizing, blocking, inhibiting, abrogating, reducing or interfering with the biological activities of CDYL2 including, for example, reduction or blocking the interaction between CDYL2 and G9a (H3K9 methyltransferase). CDYL2 antagonists include antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Antagonists also include, antagonist variants of CDYL2 protein, siRNA molecules directed to CDYL2, antisense molecules directed to CDYL2, aptamers, and ribozymes against CDYL2 protein. For instance, the CDYL2 antagonist may be a molecule that binds to CDYL2 and neutralizes, blocks, inhibits, abrogates, reduces or interferes with the biological activity of CDYL2 (such as inducing tumor cell growth and acquisition of phenotypes associated with malignant cancer progression). More particularly, the CDYL2 antagonist according to the invention is an inhibitor of CDYL2 gene expression (antisense) and small organic molecule.

By “biological activity” of CDYL2 is meant inducing tumor cell growth (through the control of cancer cell migration, invasion, stemness and EMT) and regulating the immunogenicity of cancer cells (blocking the anti-tumoral immune response).

Tests for determining the capacity of a compound to be a CDYL2 antagonist are well known to the person skilled in the art. In a preferred embodiment, the antagonist specifically binds to CDYL2 protein, CDYL2 DNA or CDYL2 mRNA in a sufficient manner to inhibit the biological activity of CDYL2. Binding to CDYL2 and inhibition of the biological activity of CDYL2 may be determined by any competing assays well known in the art. For example, the assay may consist in determining the ability of the agent to be tested as a CDYL2 antagonist to bind to CDYL2. The binding ability is reflected by the Kd measurement. The term “KD”, as used herein, is intended to refer to the dissociation constant, which is obtained from the ratio of Kd to Ka (i.e. Kd/Ka) and is expressed as a molar concentration (M). KD values for binding biomolecules can be determined using methods well established in the art. In specific embodiments, an antagonist that “specifically binds to CDYL2” is intended to refer to an inhibitor that binds to human CDYL2 polypeptide with a KD of 1 μM or less, 100 nM or less, 10 nM or less, or 3 nM or less. Then a competitive assay may be settled to determine the ability of the agent to inhibit biological activity of CDYL2. The functional assays may be envisaged such as evaluating the ability to: a) inhibit processes associated with tumor cell growth, migration, invasion, stemness and EMT and/or b) induce the expression of genes that control tumor immunogenicity, notably those involved in (or regulated by) the IFN response (see example 2 and Table 1 to 6).

The skilled in the art can easily determine whether a CDYL2 antagonist neutralizes, blocks, inhibits, abrogates, reduces or interferes with a biological activity of CDYL2. To check whether the CDYL2 antagonist binds to CDYL2 and/or is able to inhibit processes associated with tumor growth and malignant progression (for instance, tumor cell growth, migration, invasion, stemness or EMT) and/or regulation of the expression of genes involved in the anti-tumoral immune response in the same way than the initially characterized inhibitor of CDYL2 gene expression and binding assay and/or a cell proliferation assay and/or a cell migration assay and/or a cell invasion assay and/or an assay for stemness or EMT and/or an assay for regulation of genes involved in the anti-tumor immune response (such as genes involved in the IFN response or genes involved in antigen presentation and processing by MHC complex or genes involved in expression of cytokines that enhance the antitumor tumor response) may be performed with each antagonist. For instance inhibiting Interferon response can be assessed by detecting cells interferon beta with specific antibody, by GSEA (Gene Set enrichment Analysis) of IFN-response genes differentially expressed in cancer cells as described in the Examples 2 section (Table 1 to 6), or by reverse transcriptase-polymerase chain reaction assay of IFN response genes, and cell proliferation assay can be measured by CFSE-proliferation assay or migration and invasion assays as described in the Examples 1 section (FIGS. 2 and 3).

Accordingly, the CDYL2 antagonist may be a molecule that binds to CDYL2 selected from the group consisting of antibodies, aptamers, small organic molecules and polypeptides.

The skilled in the art can easily determine whether a CDYL2 antagonist neutralizes, blocks, inhibits, abrogates, reduces or interferes with a biological activity of CDYL2: (i) binding to CDYL2 (protein or nucleic sequence (DNA or mRNA)) and/or (ii) inducing tumor cell growth (or migration, invasion, stemness or EMT) and/or (iii) regulating of genes involved in the anti-tumor immune response (such as genes involved in the IFN response).

Accordingly, in a specific embodiment the CDYL2 antagonist directly binds to CDYL2 (protein or nucleic sequence (DNA or mRNA)) and promotes the expression of genes that regulate the anti-tumor immune response (such as genes involved in the IFN response).

Accordingly, in a specific embodiment the CDYL2 antagonist directly binds to CDYL2 protein, or CDYL2 DNA (gene) or CDYL2 mRNA and promotes the expression of genes that regulate the anti-tumor immune response (such as genes involved in the IFN response).

Thus in a second aspect the present invention also relates to a CDYL2 antagonist for use in a method to activate the anti-tumoral immune response of a patient affected with a cancer.

The terms “anti-tumoral immune response” means the natural ability of the immune cells to lyse cancer cells (Robbins and Kawakami, 1996, Romero, 1996).

The terms “cancer” and “tumors” refer to or describe the pathological condition in mammals that is typically characterized by unregulated cell growth, a change of cell identity and in malignant forms, the ability for invade surrounding tissues and/or give rise to metastatic tumors. More precisely, in the use of the invention, diseases, namely tumors that express CDYL2 are most likely to respond to the CDYL2 antagonist after the restoration of anti-tumor immune response (such as IFN response). In particular, the cancer may be associated with a solid tumor or lymphoma/leukemia (tumors from hematopoietic cells). Examples of cancers that are associated with solid tumor formation include breast cancer, uterine/cervical cancer, oesophageal cancer, pancreatic cancer, colon cancer, colorectal cancer, kidney cancer, ovarian cancer, prostate cancer, head and neck cancer, non-small cell lung cancer stomach cancer, tumors of mesenchymal origin (i.e; fibrosarcoma and rhabdomyoscarcoma) tumors of the central and peripheral nervous system (i.e; including astrocytoma, neuroblastoma, glioma, glioblatoma) thyroid cancer.

Preferably the cancer disease is breast cancer, colorectal cancer, lung cancer, oesophagus cancers, renal cancer, or acute myeloid leukemia.

As above mentioned CDYL2 is commonly over-expressed in breast cancer, and high CDYL2 levels correlate with poor prognosis in the ER+/HER2- and triple negative (TN) sub-types of breast cancer. High CDYL2 levels also correlate with poor prognosis in colorectal, oesophagus cancers and leukemia.

Based on the present results, the inventors propose a new therapeutic approach to prevent the emergence of cancer resistance or to treat a cancer that has developed resistance.

As mentioned, molecular and cellular changes associated with EMT and stemness in cancer cells were proposed to underlie resistance to a range of cancer therapies, as well as increased propensity to form invasive and metastatic tumors (see^{42-44; 57, 59}) Thus in a particular aspect of the present invention also relates to a CDYL2 antagonist for use in the prevention or treatment of a patient affected with a drug resistant cancer disease.

As used herein, the term “drug resistant” refers to a condition which demonstrates acquired resistance. With “acquired resistance” is meant a multifactorial phenomenon occurring in tumor formation and progression that can influence the sensitivity of cancer cells to a drug.

Acquired resistance may be due to several mechanisms such as but not limited to; alterations in drug-targets, decreased drug accumulation, alteration of intracellular drug distribution, reduced drug-target interaction, increased detoxification response, cell-cycle deregulation, increased damaged-DNA repair, and reduced apoptotic response. Several of said mechanisms can occur simultaneously and/or may interact with each other.

Various qualitative and/or quantitative methods may be used to determine if a patient has developed or is susceptible to developing a resistance to a treatment. For example, a patient who showed initial improvement while taking an antitumor drug may display signs that the antitumor drug has become less effective or is no longer effective. Symptoms that may be associated with resistance to an antitumor drug include, for example, a decline or plateau of the well-being of the patient, an increase in the size of a tumor, arrested or slowed decline in growth of a tumor, and/or the spread of cancerous cells in the body from one location to other organs, tissues or cells.

A decrease in the sensitivity of cancer cells to an antitumor drug, an increase in the growth or proliferation of cancer cells, and/or a decrease in cancer cell apoptosis as compared to a control, may also be indicative that the patient has developed or is susceptible to developing a resistance to an antitumor drug. It is possible to determine cancer cell sensitivity, growth, proliferation or apoptosis using standard methods as described further herein. For example, cancer cell sensitivity, growth, proliferation or apoptosis may be determined either in situ or in vitro.

In situ measurements may involve, for example, observing the effect of an antitumor drug therapy in a patient by examining cancer growth or metastasis. Typically, for cancer patients, RECIST criteria are analysed.

As used herein, the term “Response Evaluation Criteria In Solid Tumors (RECIST)” refers to a set of published rules that define when cancer patients improve (“respond”), stay the same (“stable”) or worsen (“progression”) during treatments. The original criteria were published in February 2000 by an international collaboration including the European Organization for Research and Treatment of Cancer (EORTC), National Cancer Institute (NCI) of the United States and the National Cancer Institute of Canada Clinical Trials Group. RECIST 1.1, published in January 2009, is an update to the original criteria. Usually, the skilled in the art concludes that the disease progresses (and hence that the patient is or is become resistant to a treatment) when at least a 20% increase in the sum of the longest diameter of target lesions, taking as reference the smallest sum longest diameter recorder since the treatment started or the appearance of one or more new lesions) by conventional methods of imaging such as computed tomography (CT).

Within the context of the invention, and according to the RECIST criteria applied to cancer patients, a patient is considered as resistant when at least a 30% increase of metastases is detected in said patient by [18F]fluoro-2-deoxy-2-d-glucose (FDG) positron emission tomography (PET) imaging (FDG-PET scan).
small organic molecule

In a particular embodiment, the CDYL2 antagonist according to the invention is a small organic molecule such as, a UNC3866 (CAS Number: 1872382-47-2) or the derived compounds such as UNC4991 (described in Barnash K. D. et al “Chromodomain Ligand Optimization via Target-Class Directed Combinatorial Repurposing” ACS Chem. Biol. 2016, 11, 2475-2483) or new compound such as 2 benzo[d]oxazol-2(3H)-one derivatives such as compound D03 which show excellent selectivity among other chromodomain proteins, including CDYL2, (described in Yang L. et al. “Identification and characterization of benzo[d]oxazol-2(3H)-one derivatives as the first potent and selective small-molecule inhibitors of chromodomain protein CDYL”, Eur J Med Chem. 2019 Nov. 15; 182:111656).

All the compound described in this article are incorporated by reference in the present invention
Inhibitor of CDYL2 gene expression

In still another embodiment, the CDYL2 antagonist is an inhibitor of CDYL2 gene expression. An “inhibitor of expression” refers to a natural or synthetic compound that has a biological effect to inhibit the expression of a gene. Therefore, an “inhibitor of CDYL2 gene expression” denotes a natural or synthetic compound that has a biological effect to inhibit the expression of CDYL2 gene.

In a preferred embodiment of the invention, said inhibitor of CDYL2 gene expression is a siRNA, an antisense oligonucleotide, a nuclease or a ribozyme.

Inhibitors of CDYL2 gene expression for use in the present invention may be based on antisense oligonucleotide constructs. Anti-sense oligonucleotides, including anti-sense RNA molecules and anti-sense DNA molecules, would act to directly block the translation of CDYL2 mRNA by binding thereto and thus preventing protein translation or increasing mRNA degradation, thus decreasing the level of CDYL2, and thus activity, in a cell. For example, antisense oligonucleotides of at least about 15 bases and complementary to unique regions of the mRNA transcript sequence encoding CDYL2 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of CDYL2 gene expression for use in the present invention. CDYL2 gene expression can be reduced by using small double stranded RNA (dsRNA), or a vector or construct causing the production of a small double stranded RNA, such that CDYL2 gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Example of commercial siRNAs against CDYL2 include, but are not limited to: esiRNA human CDYL2 (esiRNA1) (EHU042511), from Sigma Aldrich shCDYL2 #1, TRCN0000359078; shCDYL2 #2, TRCN0000130741; shCDYL2 #3, TRCN0000129278 from Sigma, Human CDYL2 shRNA in Mammalian Expression Vector (ABIN5820123) from genomics oneline, Human CDYL2 siRNAs (ID110148, ID110149, ID110150) from Thermo fisher are available.

Inhibitors of CDYL2 gene expression for use in the present invention may be based nuclease therapy (like Talen or Crispr).

The term “nuclease” or “endonuclease” means synthetic nucleases consisting of a DNA binding site, a linker, and a cleavage module derived from a restriction endonuclease which are used for gene targeting efforts. The synthetic nucleases according to the invention exhibit increased preference and specificity to bipartite or tripartite DNA target sites comprising DNA binding (i.e. TALE or CRISPR recognition site(s)) and restriction endonuclease target site while cleaving at off-target sites comprising only the restriction endonuclease target site is prevented. The guide RNA (gRNA) sequences direct the nuclease (ie Cas9 protein) to induce a site-specific double strand break (DSB) in the genomic DNA in the target sequence.

Restriction endonucleases (also called restriction enzymes) as referred to herein in accordance with the present invention are capable of recognizing and cleaving a DNA molecule at a specific DNA cleavage site between predefined nucleotides. In contrast, some endonucleases such as for example Fokl comprise a cleavage domain that cleaves the DNA unspecifically at a certain position regardless of the nucleotides present at this position. Therefore, preferably the specific DNA cleavage site and the DNA recognition site of the restriction endonuclease are identical.

Moreover, also preferably the cleavage domain of the chimeric nuclease is derived from a restriction endonuclease with reduced DNA binding and/or reduced catalytic activity when compared to the wildtype restriction endonuclease.

According to the knowledge that restriction endonucleases, particularly type II restriction endonucleases, bind as a homodimer to DNA regularly, the chimeric nucleases as referred to herein may be related to homodimerization of two restriction endonuclease subunits.

Preferably, in accordance with the present invention the cleavage modules referred to herein have a reduced capability of forming homodimers in the absence of the DNA recognition site, thereby preventing unspecific DNA binding. Therefore, a functional homodimer is only formed upon recruitment of chimeric nucleases monomers to the specific DNA recognition sites.

Preferably, the restriction endonuclease from which the cleavage module of the chimeric nuclease is derived is a type 11P restriction endonuclease. The preferably palindromic DNA recognition sites of these restriction endonucleases consist of at least four or up to eight contiguous nucleotides. Preferably, the type 11P restriction endonucleases cleave the DNA within the recognition site which occurs rather frequently in the genome, or immediately adjacent thereto, and have no or a reduced star activity. The type 11P restriction endonucleases as referred to herein are preferably selected from the group consisting of: Pvu11, EcoRV, BamH1, Bcn1, BfaSORF1835P, BfiI, Bg11, Bg111, BpuJ1, Bse6341, BsoBl, BspD6I, BstY1, Cfr101, Ec118k1, EcoO1091, EcoR1, EcoR11, EcoRV, EcoR1241, EcoR12411, HinP11, Hine11, Hind111, Hpy991, Hpy1881, Mspl, Munl, Mval, Nael, NgoMIV, Not1, OkrA1, Pab1, Pac1, PspG1, Sau3A1, Sda1, Sfi1, SgrA1, Tha1, VvuYORF266P, Dde1, Eco571, Hae111, Hha11, Hind11, and Nde1. Example of commercial gRNAs against CDYL2 include, but are not limited to: Human CDYL2 CRISPR gRNA+Cas9 in Lenti Particles (ABIN5231258) from Genomics oneline, CDYL2 CRISPR Plasmids (human) gene knockout, with CDYL2-specific 20 nt guide RNA sequences from SantaCruz Biotechnology.

Other nuclease for use in the present invention are disclosed in WO 2010/079430, WO2011072246, WO2013045480, Mussolino C, et al (Curr Opin Biotechnol. 2012 October; 23(5):644-50) and Papaioannou I. et al (Expert Opinion on Biological Therapy, March 2012, Vol. 12, No. 3: 329-342) all of which are herein incorporated by reference.

Ribozymes can also function as inhibitors of CDYL2 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of CDYL2 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Antisense oligonucleotides, siRNAs and ribozymes useful as inhibitors of CDYL2 gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide, siRNA or ribozyme nucleic acid to the cells and preferably cells expressing CDYL2. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W. H. Freeman C.O., New York, 1990) and in MURRY (“Methods in Molecular Biology,” vol. 7, Humana Press, Inc., Cliffton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, nuclease (i.e. CrispR), siRNA, shRNA or ribozyme nucleic acid sequence is under the control of a heterologous regulatory region, e.g., a heterologous promoter. The promoter may be specific for the cancer cells.
Antibody

The inventors have generated specific antibodies directed against the polypeptide CDYL2.

The polyclonal antibodies were produced by immunizing rabbits with the synthetic peptides, (CDYL2 with N-terminal 6-Histidine tagging). More precisely, the inventors have found antibodies screened for their capacity to recognize specifically the polypeptide CDYL2 (Example 2 FIGS. 9). Screening step of the antibodies of the invention has shown that these antibodies are specific of CDYL2 because they yielded a single dominant band on a western blot, which was diminished by CDYL2 RNAi and increased by CDYL2 transgenic expression, and did not cross-react with over-expressed CDYL/CDYL1 polypeptide (of the CDYL family).

In another embodiment, the CDYL2 antagonist is an antibody (the term including antibody fragment or portion) that can block the interaction of CDYL2 with histone methyltransferase G9a (H3K9 methyltransferase) and/or methyltransferase EZH2.

In preferred embodiment, the CDYL2 antagonist may consist in an antibody directed against the CDYL2, in such a way that said antibody impairs the binding of a CDYL2 to methyltransferase G9a and/or EZH2 (“neutralizing antibody”).

Then, for this invention, neutralizing antibody of CDYL2 are selected as above described for their capacity to (i) bind to CDYL2 (protein) and/or (ii) inhibiting tumor cell growth (through inhibition of cancer cell migration, invasion, stemness or EMT) and/or (iii) regulating of genes involved in the anti-tumor immune response (ie genes involved in the IFN response).

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of CDYL2. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant CDYL2 may be provided by expression with recombinant cell lines or bacteria. Recombinant form of CDYL2 may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs.

The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

As the CDYL2 is an intracellular target, the antibody of the invention acting as an activity inhibitor could be an antibody fragment without Fc fragment.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM.

IgG subclasses are also well known to those in the art and include but are not limited to human IgG1, IgG2, IgG3 and IgG4.

In another embodiment, the antibody according to the invention is a single domain antibody.

The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

The skilled artisan can use routine technologies to use the antigen-binding sequences of these antibodies (e.g., the CDRs) and generate humanized antibodies for treatment of cancer disease as disclosed herein.
Aptamer

In another embodiment, the CDYL2 antagonist is an aptamer directed against CDYL2.

Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamers of CDYL2 are selected as above described for their capacity to (i) bind to CDYL2 and/or (ii) inhibiting tumor cell growth (through inhibition of cancer cell migration, invasion, stemness or EMT) and/or (iii) regulating of genes involved in the anti-tumor immune response (ie genes involved in the IFN response).

### Method of Preventing or Treating Cancer

The present invention further contemplates a method of preventing or treating cancer disease in a subject comprising administering to the subject a therapeutically effective amount of a CDYL2 antagonist.

In one aspect, the present invention provides a method of inhibiting tumor growth in a subject comprising administering a therapeutically effective amount of a CDYL2 antagonist.

By a “therapeutically effective amount” of a CDYL2 antagonist as above described is meant a sufficient amount of the antagonist to prevent or treat a cancer disease. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicine typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The invention also relates to a method for treating a cancer in a subject having a high level of CDYL2 in a tumor sample with a CDYL2 antagonist.

The invention also relates to CDYL2 antagonist for use in the treatment of a cancer in a subject having a high level of CDYL2 in a tumor sample.

The above method and use comprise the step of measuring the level of CDYL2 protein expression (protein or nucleic sequence (DNA or mRNA)) in a tumor sample obtained from said subject wherein and compared to a reference control value.

A high level of CDYL2 is predictive of a high risk of having or developing a cancer disease (or drug resistant cancer) and means that CDYL2 antagonist could be used.

Typically, a tumor sample is obtained from the subject and the level of CDYL2 is measured in this sample. Indeed, decreasing CDYL2 levels would be particularly beneficial in those patients displaying high levels of CDYL2.

A “reference value” can be a “threshold value” or a “cut-off value”. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data (see FIG. 1). Preferably, the person skilled in the art may compare the level of CDYL2 protein expression (protein or nucleic sequence (mRNA)) of the present invention with a defined threshold value. In one embodiment of the present invention, the threshold value is derived from the CDYL2 protein level (or ratio, or score) determined in a tumor sample derived from one or more subjects who are responders (to the method according to the invention). In one embodiment of the present invention, the threshold value may also be derived from CDYL2 protein level (or ratio, or score) determined in a skin sample derived from one or more subjects or who are non-responders. Furthermore, retrospective measurement of the CDYL2 protein level (or ratio, or scores) in properly banked historical subject samples may be used in establishing these threshold values.

For example, after determining the expression level of the CDYL2 protein expression (protein or nucleic sequence (mRNA)) in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the method of the invention comprises the use of a classification algorithm typically selected from Linear Discriminant Analysis (LDA), Topological Data Analysis (TDA), Neural Networks, Support Vector Machine (SVM) algorithm and Random Forests algorithm (RF). In some embodiments, the method of the invention comprises the step of determining the subject response using a classification algorithm. As used herein, the term “classification algorithm” has its general meaning in the art and refers to classification and regression tree methods and multivariate classification well known in the art such as described in U.S. Pat. No. 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

In some embodiments, the method of the present invention comprises a) quantifying the level of CDYL2 protein expression (protein or nucleic sequence (mRNA)) in the tumor sample; b) implementing a classification algorithm on data comprising the quantified CDYL2 protein so as to obtain an algorithm output; c) determining the probability that the subject will develop a cancer disease (or drug resistant cancer) from the algorithm output of step b).

The algorithm used with the method of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data.

Generally, a computer will also include, or be operatively coupled to receive data from or transfer data to, or both, one or more mass storage devices for storing data, e.g., magnetic, magneto-optical disks, or optical disks. However, a computer need not have such devices.

Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

“Risk” in the context of the present invention, relates to the probability that an event will occur over a specific time period, as in the conversion to cancer disease (or drug resistant cancer), and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no conversion. Alternative continuous measures, which may be assessed in the context of the present invention, include time to cancer disease (or drug resistant cancer) conversion risk reduction ratios.

“Risk evaluation,” or “evaluation of risk” in the context of the present invention encompasses making a prediction of the probability, odds, or likelihood that an event or disease state may occur, the rate of occurrence of the event or conversion from one disease state to another, i.e., from a normal condition to a cancer disease (or drug resistant cancer) condition or to one at risk of developing a cancer disease (or drug resistant cancer). Risk evaluation can also comprise prediction of future clinical parameters, traditional laboratory risk factor values, or other indices of cancer disease (or drug resistant cancer), such as cellular population determination in peripheral tissues, in serum or other fluid, either in absolute or relative terms in reference to a previously measured population. The methods of the present invention may be used to make continuous or categorical measurements of the risk of conversion to cancer disease (or drug resistant cancer), thus diagnosing and defining the risk spectrum of a category of subjects defined as being at risk for a cancer disease (or drug resistant cancer). In the categorical scenario, the invention can be used to discriminate between normal and other subject cohorts at higher risk for cancer disease (or drug resistant cancer). In other embodiments, the present invention may be used so as to help to discriminate those having cancer disease (or drug resistant cancer) from normal.

In another aspect, the present invention provides a method to activate the anti-tumoral immune response of a patient affected with a cancer comprising administering to the subject a therapeutically effective amount of Chromodomain on Y-like 2 (CDYL2) antagonist/inhibitor of CDYL2 gene expression.

In a still another aspect, the present invention provides a method of preventing development of a drug resistant cancer in a subject comprising administering to the subject a therapeutically effective amount of amount of Chromodomain on Y-like 2 (CDYL2) antagonist/inhibitor of CDYL2 gene expression.

### Pharmaceutical Compositions of the Invention

The CDYL2 antagonist/inhibitor of CDYL2 gene expression as described above may be combined with pharmaceutically acceptable excipients, and optionally sustained-release matrices, such as biodegradable polymers, to form therapeutic compositions.

Accordingly, the present invention relates to a pharmaceutical composition comprising a CDYL2 antagonist according to the invention and a pharmaceutically acceptable carrier.

The present invention also relates to a pharmaceutical composition for use in the prevention or treatment of cancer disease and drug resistant cancer comprising a CDYL2 antagonist according to the invention and a pharmaceutically acceptable carrier.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In therapeutic applications, compositions are administered to a patient already suffering from a disease, as described, in an amount sufficient to cure or at least partially stop the symptoms of the disease and its complications. An appropriate dosage of the pharmaceutical composition is readily determined according to any one of several well-established protocols. For example, animal studies (for example on mice or rats) are commonly used to determine the maximal tolerable dose of the bioactive agent per kilogram of weight. In general, at least one of the animal species tested is mammalian. The results from the animal studies can be extrapolated to determine doses for use in other species, such as humans for example. What constitutes an effective dose also depends on the nature and severity of the disease or condition, and on the general state of the patient's health.

In therapeutic treatments, the antagonist contained in the pharmaceutical composition can be administered in several dosages or as a single dose until a desired response has been achieved. The treatment is typically monitored and repeated dosages can be administered as necessary. Compounds of the invention may be administered according to dosage regimens established whenever inactivation of CDYL2 is required.

The daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 10 mg/kg of body weight per day. It will be understood, however, that the specific dose level and frequency of dosage for any particular patient may be varied and will depend upon a variety of factors including the activity of the specific compound employed, the metabolic stability, and length of action of that compound, the age, the body weight, general health, sex, diet, mode and time of administration, rate of excretion, drug combination, the severity of the particular condition, and the host undergoing therapy.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The appropriate unit forms of administration include forms for oral administration, such as tablets, gelatine capsules, powders, granules and solutions or suspensions to be taken orally, forms for sublingual and buccal administration, aerosols, implants, forms for subcutaneous, intramuscular, intravenous, intranasal or intraocular administration and forms for rectal administration.

In the pharmaceutical compositions of the present invention, the active principle is generally formulated as dosage units containing from 0.5 to 1000 mg, preferably from 1 to 500 mg, more preferably from 2 to 200 mg of said active principle per dosage unit for daily administrations.

When preparing a solid composition in the form of tablets, a wetting agent such as sodium laurylsulfate can be added to the active principle optionally micronized, which is then mixed with a pharmaceutical vehicle such as silica, gelatine, starch, lactose, magnesium stearate, talc, gum arabic or the like. The tablets can be coated with sucrose, with various polymers or other appropriate substances or else they can be treated so as to have a prolonged or delayed activity and so as to release a predetermined amount of active principle continuously.

A preparation in the form of gelatin capsules is obtained by mixing the active principle with a diluent such as a glycol or a glycerol ester and pouring the mixture obtained into soft or hard gelatine capsules.

A preparation in the form of a syrup or elixir can contain the active principle together with a sweetener, which is preferably calorie-free, methyl-paraben and propylparaben as an antiseptic, a flavoring and an appropriate color.

The water-dispersible powders or granules can contain the active principle mixed with dispersants or wetting agents, or suspending agents such as polyvinyl-pyrrolidone, and also with sweeteners or taste correctors.

Rectal administration is effected using suppositories prepared with binders which melt at the rectal temperature, for example cacao butter or polyethylene glycols.

Parenteral, intranasal or intraocular administration is effected using aqueous suspensions, isotonic saline solutions or sterile and injectable solutions which contain pharmacologically compatible dispersants and/or wetting agents, for example propylene glycol, butylene glycol, or polyethylene glycol.

Thus a cosolvent, for example an alcohol such as ethanol or a glycol such as polyethylene glycol or propylene glycol, and a hydrophilic surfactant such as Tween® 80, can be used to prepare an aqueous solution injectable by intravenous route. The active principle can be solubilized by a triglyceride or a glycerol ester to prepare an oily solution injectable by intramuscular route.

Transdermal administration is effected using multilaminated patches or reservoirs into which the active principle is in the form of an alcoholic solution.

Administration by inhalation is effected using an aerosol containing for example sorbitan trioleate or oleic acid together with trichlorofluoromethane, dichlorotetrafluoroethane or any other biologically compatible propellant gas.

The active principle can also be formulated as microcapsules or microspheres, optionally with one or more carriers or additives.

Among the prolonged-release forms which are useful in the case of chronic treatments, implants can be used. These can be prepared in the form of an oily suspension or in the form of a suspension of microspheres in an isotonic medium.

The active principle can also be presented in the form of a complex with a cyclodextrin, for example .alpha.-, .beta.- or .gamma.-cyclodextrin, 2-hydroxypropyl-.beta.-cyclodextrin or methyl-.beta.-cyclodextrin.

### FIGURES

FIG. 1: High CDYL2 expression level in breast cancer is associated with poor prognosis (A) CDYL2 mRNA expression in breast tumors compared to normal tissues, as derived from the Oncomine database and GEO2R analysis of the indicated GEO datasets. (B) Paired analysis of CDYL2 mRNA (TCGA, RNA-seq) and protein levels (CPTAC, mass spectrometry) in individual tumor specimen. (C-D) Kaplan-Meier overall survival (OS) analysis performed from TCGA breast cancer subtypes: ER+/HER2- (C); and triple negative (TN) (D) using best cutoff of CDYL2 expression (High and Low). Significance using LogRank p-value and Hazard Ratio (CI).

FIG. 2: CDYL2 over-expression in the non-invasive breast cancer line MCF7 induces transcriptional changes associated with malignant progression

(A) Western blot analysis of CDYL2 and beta-Actin expression in MCF7-CDYL2 and MCF7-Vector cells. (B) Volcano plot showing genes UP- or DOWN-regulated at least 2.5-fold at an adjusted p-value less than 0.05. (C) Selected molecular signatures over-represented in either the UP- or DOWN-regulated gene sets from (B). (D) qRT-PCR validation of selected differentially expressed genes from (C). Mean of three independent experiments±S.D. Significant at p<0.05 (T-test).

FIG. 3: CDYL2 over-expression in MCF7 cells induces EMT-like changes, accompanied by increased migration, invasiveness and mammosphere formation

(A) qRT-PCR analysis of EMT markers, normalization to GAPDH. Mean±S.D of three experiments. Significant at p<0.05 (T-test). (B) Western blot analysis of an EMT markers, ER-alpha, CDYL2 and Beta-actin. (C) Diagram of the xCELLigence quantitative, real-time migration and invasion assay. (D) Relative migration efficiency of MCF7-Vector and MCF7-CDYL2 cells. Both (D) and (E) show technical quadruplicates+/−S.D. Repeated at least three times with similar results. (E) Invasion assays were performed as in (D), except that the porous membrane separating the upper and lower chambers of the transwell was first overlaid with Matrigel. (F) Zebrafish embryo cell invasion and migration assay. Shown Percentage of embryos exhibiting tail metastases illustrating the metastasis of fluorescently labelled MCF7-CDYL2 or MCF7-Vector cells from the site of injection to the tail. Repeated three times with similar results. (G) Mammosphere formation in MCF7-CDYL2 cells compared to MCF7-Vector controls. The indicated number of ells were plated in 96-well plates. Mammospheres with size >50 m were counted after 8 days. Assay was repeated three times with similar results. T-test: **** p<0.0001. (H) Mammospheres diameter analysis. Shown is mean+S.D. of eight wells in which 1000 cells were seeded. T-test: *p<0.05. (I-J) FACS analysis of CD44 and CD24 expression. Shown are representative scatter plots (I) and the mean of three independent experiments +/−S.D. (J). T-test: *p<0.05.

FIG. 4: RNAi knockdown of CDYL2 in the invasive breast cancer cell line MDA-MB-231 induces transcriptional and phenotypic changes associated with inhibition of malignancy.

(A-B) CDYL2 knock-down validated by RT-qPCR (A) and western blotting (B). (C) Volcano plot showing genes Up- or Down-regulated at least 1.25-fold. (D) Selected GSEA analysis of Up- or Down-regulated genes from (C). (E) qRT-PCR validation of selected genes from (D), normalized to GAPDH. Shown if mean±S.D. of three independent experiments. All significant p<0.05 (T-test). (F) Western blot analysis of a panel of EMT markers, CDYL2 and Beta-actin. (G) Relative migration efficiency of MDA-MB-231 cells treated with esiLuc or esiCDYL2 (esiCD2). Both (G) and (H) show technical quadruplicates+/−S.D. Repeated three times with similar results. (H) Invasion assays were performed as in (G), except measuring migration across a microporous membrane overlaid with Matrigel. (I) Zebrafish embryo cell invasion and migration assay. Shown are micrographs. Percentage of embryos exhibiting tail metastases illustrating the metastasis of fluorescently labelled MDA-MB-231 cells treated with esiLuc or esiCDYL2 from the site of injection to the tail. Repeated three times with similar results. (J) Mammosphere formation in MDA-MB-231 cells treated with esiLuc or esiCDYL2. The indicated number of ells were plated in 96-well plates. Mammospheres with size >50 m were counted after 8 days. Assay was repeated three times with similar results. T-test: **** p<0.0001. (K) Mammospheres diameter analysis. Shown is mean+S.D. of eight wells in which 1000 cells were seeded. T-test: *p<0.05. (K-M) FACS analysis of CD44 and CD24 expression. Shown are representative scatter plots (L) and the mean of three independent experiments+/−S.D. (M). T-test: *p<0.05.

FIG. 5: CDYL2 regulation of NF-κB and STAT3 signaling contributes to its induction of invasion and mammosphere formation

(A) Selected GSEA signatures enriched in the indicated RNA-seq datasets. (B) Western blot of Ser536-phosphorylated p65 and Tyr705-phosphorylated STAT3 in MCF7-Vector versus MCF7-CDYL2. The levels of total p65, STAT3 and 3-actin were also probed. (C) As for (B), except comparing MDA-MB-231 cells treated with esiLuc or esiCDYL2. (D,E) Western blot validation of RNAi knockdown of p65 (D) or STAT3 (E) in MCF7-Vector and MCF7-CDYL2 cells. β-actin, loading control. (F) qRT-PCR analysis of the effect of RNAi knockdown of p65 on the expression of a panel of NF-κB target genes that were up-regulated in MCF7-CDYL2 compared to MCF7-Vector cells. Shown is mean of three experiments ±S.D. T-test, p<0.05. (G) As in (F), except the effect of RNAi knockdown of STAT3 was evaluated. (H,I) Invasion assays of MCF7-CDYL2 in MCF7 cells treated with either control RNAi or siRNA targeting p65 (H) or STAT3 (I). Assay was repeated three times with similar results. Shown is mean+/−S.D. of quadruplicate readings. (J,K) Mammospheres assay of MCF7-CDYL2 in MCF7 cells treated with either control RNAi or siRNA targeting p65 (J) or STAT3 (K). Mammospheres from 1000 seeded cells with size >50 m were counted after 8 days. Shown is the median+/−S.D. of three experiments. T-test: ** n<0.001: *** n<0.0001.

FIG. 6: Identification of miR-124 as a candidate mediator of CDYL2 regulation of STAT3 and NF-kB signaling

(A) CDYL2 ChIP-seq peaks upstream of the MIR124 genes in MCF7-Vector cells (top graph) and MCF7-CDYL2 (lower graph). (B) ChIP-qPCR validation of the MIR124-proximal CDYL2 peaks represented in (A). A non-specific antibody (IgG) was used as negative control. All ChIP-qPCRs (B,C) show the mean enrichment as a percentage of Input of three experiments, ±S.D. T-test: * p<0.05; ** p<0.01. (C) As in (B), except CDYL2 or IgG ChIP was performed using chromatin prepared from MDA-MB-231 cells treated with esiLuc or esiCDYL2. (D) Selected GSEA signatures enriched in the indicated RNA-seq datasets. (E) qRT-PCR analysis of pre-mir-124 and miR-124-3p levels in MCF7-CDYL2 and MCF7-Vector cells. Expression was normalized to an unrelated miRNA. Data represent the mean of three independent experiments±S.D. T-test: * p<0.05; ** p<0.01. (F) As in (E), except qRT-PCR analysis was performed using miRNA prepared from MDA-MB-231 cells treated with esiLuc or esiCDYL2. (G) qRT-PCR analysis of the expression of miR-124-3p target genes in MCF7-CDYL2 and MCF7-Vector cells. Shown is mean±S.D. of three experiments. T-test: * p<0.05. (H) As in (G), except qRT-PCR analysis was performed using RNA prepared from MDA-MB-231 cells treated with esiLuc or esiCDYL2. (I) Western blot of phosphorylated p65 (Ser 536), Total p65, phosphorylated STAT3 (Tyr 705), and total STAT3 in MCF7-CDYL2 cells treated with a miR124-3p mimic or miR control. Repeated three times with similar results. (J) Western blot of phosphorylated p65 (Ser 536), Total p65, phosphorylated STAT3 (Tyr 705), and total STAT3 in MDA-MB-231 cells co-treated with esiCDYL2 and either an anti-miR-124-3p oligonucleotide, or a control anti-miR. Repeated three times with similar results.

FIG. 7: CDYL2 interaction with G9a, GLP, EZH2 and SUZ12, and its regulation of G9a, EZH2, H3K9me2 and H3K27me3 levels upstream of MIR124 genes.

(A) Immunoblots of immunoprecipitates of non-specific IgG, CDYL2, EZH2 and G9a from MCF7 cell lysates. Repeated three times with similar results. (* specific band; ** IgG heavy chain). (B) ChIP-qPCR analysis of the relative occupancy of CDYL2, EZH2 and G9a upstream of MIR124 genes in MCF7-CDYL2 and MCF7-Vector cells. IgG, negative control ChIP. qPCR analysis was also performed at an unrelated negative control sequence. Shown is the mean enrichment as a percentage of Input of three independent experiments, ±S.D. T-test (* p<0.05; ** p<0.01; *** p<0.001). (C) As in (B), except using antibodies specific to H3K9me2, H3K27me3, H3 and IgG. These ChIP analyses were conducted using the same lysates as in (B), so are paired analyses. (D,E) Experiments were conducted as described in (B) and (C), except using chromatin lysates prepared from MDA-MB-231 cells treated with esiCDYL2 or esiLuc. (F) Schematic model of the proposed contribution of CDYL2 to epigenetic regulation of MIR124, cell signaling, and malignancy-associated cellular processes.

FIG. 8 High CDYL2 expression level in a variety of human cancer and is associated with poor prognosis.

(A) Oncomine analysis cancer cohorts revealed upregulation of CDYL2 mRNA in breast, colorectal, esophagus cancers and leukemia, but a downregulation in lymphoma. (B,C) Kaplan-Meier overall survival (OS) analysis performed from TCGA colorectal cancer (B), or rectal adenocarcinoma (C). High or low CDYL2 mRNA was based best cutoff. (D,E) Kaplan-Meier overall survival (OS) analysis performed from TCGA lung squamous cell carcinoma (D) or lung adenocarcinoma (E). High or low CDYL2 mRNA was based respectively on best cutoff of and highest versus lowest quartiles. Significance using LogRank p-value and Hazard Ratio (CI) are indicated.

FIG. 9: Anti-CDYL2 antibody validation

(A) Immunoblots (IB) of whole cell extract from MCF-7 show that CDYL2 antiserum reacts with one band of the expected molecular weight. (B) RNAi of CDYL2 in MCF-7 and HEK293T cells diminished the anti-CDYL2 immunoblot band intensity. (C) Transfection of MCF-7 with an HA-CDYL2 expression plasmid, but not empty vector, increased the anti-CDYL2 IB band intensity. Anti-HA blotting confirmed that the transfected and endogenous proteins co-migrate on SDS-PAGE gels. (D) Transfection of HEK293T cells with HA-CDYL (CDYL1) expression plasmids confirmed the CDYL2 antiserum does not cross-react with CDYL, which migrated at a higher molecular weight. HA blotting indicates the position of the CDYL band.

FIG. 10: Examples of CT scans of the lungs of mice CT scans of the lungs of mice illustrating the numerous tumours visible in the shControl MDA-MB-231 treated group (A) (visible as black spots against the light background of the lung tissue). By contrast, the shCDYL2 #1 (B) and shCDYL2 #2 (C) groups had very few tumours per lung.

### EXAMPLE 1: IDENTIFICATION OF CDYL2 AS THERAPEUTIC TARGET IN CANCER

Material & Methods

Cell Culture

MCF7 cells (ATCC, HTB-22) and their derivatives were grown in DMEM Low Glucose (Gibco, 31885-023) supplemented with 10% of FBS (Gibco, 10270-106), 40 μg/mL of gentamicin (Gibco, 15710-049) and 0.6 μg/mL of insulin (NovoRapid, 3525909). MDA-MB-231 cells (ATCC, HTB-26) were grown in DMEM GlutaMAX (Gibco, 10566016) supplemented with 10% of FBS (Gibco, 10270-106), 1% penicillin/streptomycin (Gibco, 15140122). Cells were grown at 37° C. and 5% C02 in a humidified incubator and passaged every 2-4 days by trypsinization. Sustained expression of ER-alpha in MCF7 was validated regularly by western blotting and immunofluorescence. Cells were regularly tested for mycoplasma using a commercial kit (ATCC, 30-1012K), and cultures renewed from low passage stocks every two months or less.

### Stable Expression of CDYL2 in MCF7 Cells

CDYL2 cDNA was cloned by PCR from an MCF7 cDNA library using primers and Phusion polymerase (NEB, M0530), and inserted into the Gateway pENTR-D-TOPO vector (Invitrogen, K240020). Sequencing on both strands confirmed that the cDNA corresponded to a published CDYL2 sequence (Genbank, NM_152342.2). The cloned cDNA was then transferred into MSCV plasmid (Addgene #41033) using LR Clonase (Invitrogen, 11791100), and the resulting expression construct validated by sequencing. MCF7 were then stably transduced with MSCV (Vector) or MSCV-CDYL2 retroviruses and selected for 14 days using 2 μg/mL puromycin (Sigma, P8833). Expression was confirmed by western blotting and immunofluorescence using CDYL2 antibody.

RNA Interference and microRNA Treatments

For transient RNAi, MDA-MB-231 cells were transfected with CDYL2 esiRNA (Sigma, EHU042511), esiLuciferase (Sigma, EHUFLUC), on-target plus p65 siRNA (Dharmacon, L-003533-00), or on-target plus STAT3 siRNA (Dharmacon, L-003544-00-0005) or on-target plus control siRNA (Dharmacon, D-001810-01-05) using Interferin reagent (Polyplus, 409-10) according to the manufacturer's instructions. Cellular assays and analysis were performed between 48 and 72 h post-transfection, according to the experiment. For stable RNAi, MCF7 cells were transduced with non-targeting control shRNA pLKO.1 lentiviruses (Sigma, SHC016) or shRNA-pLKO targeting CDYL2 (Sigma, shCDYL2 #1, TRCN0000359078; shCDYL2 #2, TRCN0000130741; shCDYL2 #3, TRCN0000129278), selected for 14 days using 2 μg/mL puromycin (Sigma, P8833). Cellular assays and analysis were then performed between weeks 2 and 4 post-transduction. The Hsa-miR-124-3p MISSION microRNA Mimic (Sigma, HMI0086) and its corresponding miRNA mimic Negative Control (Sigma, HMC0002) were transfected into MCF7-Vector or MCF7-CDYL2 cells using interferin. Samples were harvested for analysis 48-72 h post-transfection. The hsa-miR-124-3p Inhibitor (Qiagen, YT04102198-ADA), and its corresponding negative control (Qiagen, YT00199006-ADA) were co-transfected into MDA-MB-231 cells along with either esiCDYL2 or esiLuciferase siRNA, using Interferin reagent. Samples were harvested 72 h later for analysis.

### Antibodies and Reagents

The following antibodies were used: CDYL2 (Sigma, HPA041016), ERa (Santa Cruz, sc-8002), β-Actin-HRP (Sigma, A3854), Vimentin (Dako, M0725), E-cadherin (BD 610682), Snail/Slug (Abeam, ab85936), Twist (Abeam, ab50887), Phospho-NF-κB p65 (Ser536) (Cell Signaling Technologies, #3031), total p65 (Cell Signaling Technologies, #3034), phosphor-STAT3 (Tyr705) (Cell Signaling Technologies, #9145), total STAT3 (Cell Signaling Technologies, #9139), CD44-FITC (Miltenyl Biotec, 130-113-341), CD24-PE (Miltenyl Biotec, 130-095-953), EZH2 (Cell Signaling Technologies #5246S), ChIP-grade EZH2 (Diagenode, C15410039), SUZ12 (Cell Signaling Technologies #3737S), H3K9me2 (Abeam, Ab1220), H3K27me3 (Diagenode, C15410069), rabbit IgG (Bethyl, P120-101), H3 (Abeam, Ab1791). The G9a and GLP antibodies were gifts from Y. Nakatani lab (PMID: 12004135).

### Immunoblot

Cells were washed with PBS, lysed in ice-cold RIPA buffer (10 mM Tris-Cl (pH 8.0), 1 mM EDTA, 1% Triton X-100, 0.1% sodium deoxycholate, 0.1% SDS, 140 mM NaCl, 1 mM PMSF, all from Sigma) containing protease inhibitor cocktail (Roche, 04693132001) phosphatase inhibitors cocktail (Roche, 4906845001), and sonicated briefly. Cleared lysates were resolved on Bis-Tris NuPage Gels (Invitrogen), transferred to Nitrocellulose (GE Healthcare), and probed according to the primary antibody manufacturer's protocols. Images were collected using the ChemiDoc system (BioRad).

### Co-Immunoprecipitation

Cells were grown to sub-confluence in 15 cm tissue culture treated plates (Corning). Monolayers were washed three times with ice-cold PBS, scraped in cold PBS containing a protease inhibitor cocktail (Roche) and pelleted. The resulting cell pellets were lysed in lysis buffer (50 mM Tris pH8; 150 mM NaCl; 1% NP40; 2 mM EDTA) containing protease inhibitor cocktail (Roche, 04693132001) phosphatase inhibitors cocktail (Roche, 4906845001). The lysates mechanically homogenized using 25G syringes (BD, #300600), then incubated for 30 min at 4° C. with rotation. Lysates were centrifuged at 12000 rpm 15 min 4° C. to clear debris, treated with DNase I (Qiagen, #79254) and RNase A (Sigma, R4875), then precleared with protein A agaroses beads (for 1 hour 4° C. with rotation. Immunoprecipitation was performed by incubating indicated antibodies with the lysates overnight at 4° C. with rotation, the prewashed protein A agaroses beads were incubated with the lysates for 2 hours 4° C. with rotation. The beads were washed 5 times with wash buffer (10 mM Tris pH8; 1 mM EDTA; 1 mM EGTA; 150 mM NaCl; 1% Triton) containing protease inhibitor cocktail (Roche, 04693132001) phosphatase inhibitors cocktail (Roche, 4906845001). The immune-precipitated beads were boiled in Laemmli buffer and then subjected to immunoblotting.

### Immunofluorescence

Cells were seeded on sterilized coverslips. Forty-eight hours after seeding, cells were fixed with 4% paraformaldehyde for 15 min at room temperature. Fixed cells were permeabilized by NP-40 0.5% at room temperature for 15 min and blocked with 5% FBS in PBS+0.1% NP-40 at room temperature for 1 h. Cells were then probed with the indicated primary antibodies 1 h at room temperature and then Alexa dye tagged secondary antibody as mentioned for 1 h at room temperature. Coverslips were mounted using Dapi mounting medium (Vectashield; VECTOR Laboratories) and observed under the upright microscope (ZEISS AXIOIMAGER, SIP 60549), images were analyzed using Zen software.

### Flow Cytometry

For CSC analysis, the cells were labeled with anti-CD44-PerCP-Cy 5.5 and anti-CD24-PE antibodies according to the manufacturer's instructions. All analyses were performed using a BD FACSCalibur flow cytometer and BD CellQuest software (BD Biosciences).

### Gene Expression

RNA was extracted using TRI-reagent (Sigma, T9424), and cDNA synthesized using the High-Capacity cDNA Reverse Transcription Kit (Applied Biosystems, #4368814). miRNA was extracted using the miRNeasy Mini Kit (Qiagen, #217004) following the manufacturer's instructions, and cDNA synthesized using miScript II RT Kit (Qiagen, #218161). The RT-qPCR was performed using the Fast SYBR Green 2× Master Mix (Applied Biosystems, #4385610) and an LC480 PCR machine (Roche).

### RNA-Seq and Enrichment Analysis

RNA libraries were prepared with the TruSeq Stranded Total-RNA kit and sequenced on a Illumina NextSeq sequencing machine. After careful quality controls, raw data were aligned on the human genome (hg38) with STAR v2.7.0f (Dobin et al., 2013) and default parameters. Read counts on each genes of the Gencode annotation v29 were produced by STAR.

Unless otherwise specified, the analyses were performed using R (v 3.4.4) and illustrations produced with the ggplot2 (Wickham, H., (2016) Elegant Graphics for Data Analysis) and ggpubr packages. Starting from raw counts, we used the R package DESeq2 (Love et al, 2014) (v1.14) to perform the differential expression analyses. Each time the design was set as ˜REP +TYPE, where REP refers to the replicate number (paired analysis) and TYPE to the treatment group. Differential expression was tested using the Wald test, and p-values were corrected with the Benjamini-Hochberg method. To test the pathway enrichment of a list of genes, we used the R packages clusterProfiler (Yu et al, 2012) (v 3.8.1), msgidbr (v 6.2.1), org.Hs.eg.db (v 3.5.0) and reactomePA (v 1.24) (Carlson, M, 2013). We tested the list of genes against pathways from msigdb hallmarks, GO molecular functions, KEGG and Reactome. Over-representation p-values were corrected with the Benjamini-Hochberg method.

### Chromatin Immunoprecipitation

For MDA-MB-231 ChIP-qPCR analysis, cells grown in 6-well plates were treated as described, then cross-linked by the addition of 1% methanol-free formaldehyde to the cell culture medium for 10 minutes, followed by the addition of glycine to a final concentration of 125 mM for 5 minutes. Monolayers were washed three times with ice-cold PBS, scraped in cold PBS containing a protease inhibitor cocktail (Roche) and pelleted. Chromatin lysates were prepared from the cross-linked pellets, and ChIP assays performed using a commercial kit (Cell Signaling Technologies, #9003), with the indicated antibodies. Quantitative PCR was performed. Input chromatin was purified in parallel in each ChIP assay and used to determine the percentage of input recovered in each ChIP assay.

For MCF7 ChIP-seq and ChIP-qPCR analysis, cells were grown to sub-confluence in 15 cm tissue culture treated plates (Corning), cross-linked, washed and pelleted as described for MDA-MB-231 cells, above. The resulting cell pellets were pre-treated by incubating in lysis buffer A (Lysis Buffer 1 (50 mM HEPES pH 7.5; 140 mM NaCl, 1 mM EDTA, 10% glycerol, 0.5% NP-40, 0.25% Triton X-100, 1× protease inhibitors) for 10 minutes at 4° C., then lysis buffer B (10 mM Tris-HCl pH 8.0; 200 mM NaCl; 1 mM EDTA; 0.5 mM EGTA; 1× protease Inhibitors) for 10 minutes at room temperature, as previously described (Lee et al., 2006). They were then lysed in buffer C (10 mM Tris-HCl, pH 8.0, 100 mM NaCl, 1 mM EDTA, 0.5 mM EGTA, 0.1%; Na-Deoxycholate, 0.5% SDS, 1× protease inhibitors), incubated on ice for 30 minutes with occasional vortexing, then sonicated on ice to an average fragment size of 150 bp using a Branson sonicator. The sonicated lysate was centrifuged at 12,000 r.p.m. in a benchtop centrifuge at 4° C., the supernatant diluted five times in buffer C, and 1 mL aliquots of this added to 50 μL of magnetic protein A beads (Invitrogen) pre-coated with 5 μg of anti-CDYL2 IgG. These were incubated overnight at 4° C. with rotation, then pelleted and washed with two sequential additions each of wash buffer 1 (0.1% SDS, 1% Triton X-100, 2 mM EDTA, 20 mM Tris-HCl, pH 8, 150 mM NaCl), wash buffer 2 (0.1% SDS, 1% Triton X-100, 2 mM EDTA, 20 mM Tris-HCl, pH 8, 500 mM NaCl), wash buffer 3 (0.25M LiCl, 1% NP40, 1% deoxycholate, 1 mM EDTA, 10 mM Tris-HCl, pH 8), and TE buffer (10 mM Tris-HCl, 1 mM EDTA, pH 8.0, 50 mM NaCl). Chromatin was eluted by incubating for 30 minutes at 65° C. in elution buffer (50 mM Tris-HCl pH 8, 10 mM EDTA pH 8, 1% SDS) with frequent vortexing. Crosslinks were reversed by overnight incubation at 65° C., and eluates treated with RNAse A (Sigma, (Yu and He, 2016)) for 2 h, followed by Proteinase K for 2 h, then extracted using a classical Phenol-Chloroform/Ethanol precipitation protocol.

### ChIP-seq

Pair end DNA sample libraries were sequenced using Illumina. Raw sequences were aligned to human genome hgl9, using Bowtie 2.0 (Langmead and Salzberg, 2012) with paired-end parameters. Normalized and subtraction bigwig files were obtaining using deepTools (Ramirez et al., 2016). Analysis of ChIP-Seq data was in the galaxeast.fr instance. Significant peaks were called using MACS2 (Zhang et al., 2008). Called peaks were annotated using Homer_AnnotatePeaks.

### Colony Formation Assay

To form adherent colonies, 3000 cells were seeded in 12 well tissue-culture treated plates. After 14 days of growth, all colonies were stained with crystal violet solution (crystal violet 0.05%, formaldehyde 1%, methanol 1%) for 20 min, washed extensively with water. Colonies were counted and their size was measured using Fiji software.

Migration and invasion assays Real-time cell migration and invasion were measured using the xCELLigence RTCA DP apparatus (Acea Biosciences, Inc.) according to the manufacturer's protocol. Briefly, 40,000 cells were prepared in serum-deprived medium and then added to the upper chambers of CIM-16 plates (Aceabio, #5665817001). Complete medium containing 10% FBS was added to the lower chamber. For invasion assays, Matrigel diluted in serum-free medium at 500 μg/mL (BD Biosciences, #354234) was added to the upper chamber and allowed to set before adding cells. Migration across the membrane separating the two chambers was expressed as the Cell Index (CI).

### Mammosphere Formation Assay

Cells were seeded at several densities (10, 50, 100, 1000 cells per well) in 96-well ultra-low attachment plates (Corning, #3474) with MEBM Basal Medium (Lonza, CC-3151) containing 2% B-27 (Invitrogen, 17504-044), 20 ng/mL EGF (Sigma, E9644), 4 μg/mL insulin (Novo Nordisk, #3525909), and 2 μg/mL hydrocortisone (Sigma, H0135). Mammospheres were cultured for 1-2 weeks, with image collection approximately every three days starting at day 8. Whole-well images were taken with the IncuCyte ZOOM System (Essen Bioscience) using a 4× phase contrast objective. Mammosphere diameter and the number of mammospheres >50 μm were determined by image analysis using Fiji software (Fiji).

### Zebrafish Embryo Metastasis Assay

Zebrafish embryos were raised under standard experimental conditions. Cells trypsinated, resuspended in serum-free media, and stained with lipophilic dyes DiO or DiD from the Vybrant Multicolor Cell-Labeling Kit (Invitrogen, V22889) for 20 minutes at 37° C., then washed resuspended in PBS 1×. 48 hours post-fecundation, the embryos were dechorionated and anesthetized with tricaine (Sigma-Aldrich, E10521). The anesthetized embryos were subjected to microinjection. 20 nl of cell suspension, which represent approximately 300 labeled human cells, were injected into perivitelline space of each embryo. The injected zebrafish embryos were immediately placed at 30° C. for 24 hours in presence of N-phenylthiourea (Sigma-Aldrich, P7629) to inhibit melanocyte formation. For metastasis assessment, the anesthetized embryos were evaluated using the fluorescent microscope Axio Observer Zeiss microscope (Zeiss).

### Statistical Analysis

CDYL2 expression levels from TCGA were stratified based on molecular markers such as ERa and HER2 expression by IHC. Correlation analysis between CDYL2 RNA and protein levels were performed using GraphPad Prism7. For overall survival analysis, patients were divided into two groups (low and high CDYL2) using the expression level of CDYL2 and best cutoff. Kaplan-Meier survival plots, log-rank p-values, hazard ratios were calculated using GraphPad Prism7.

### Results

High CDYL2 Expression Level in Breast Cancer is Associated with Poor Prognosis

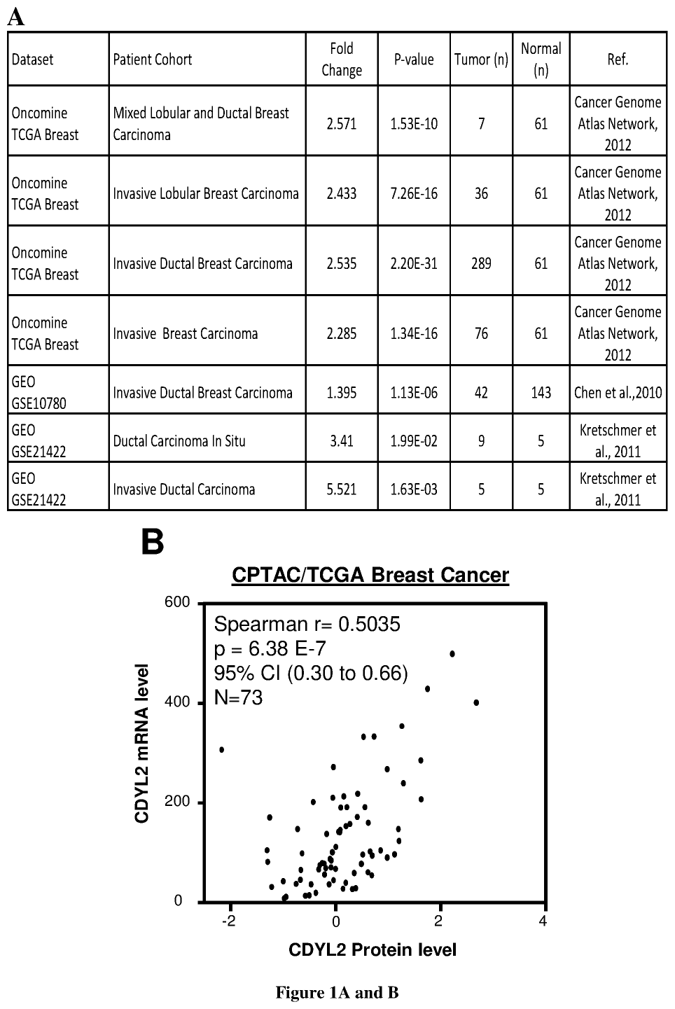

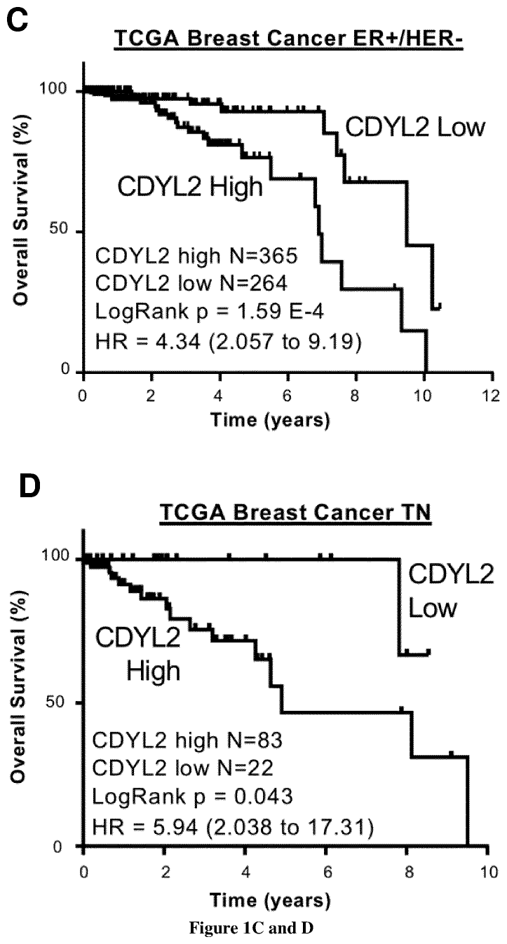

Datamining revealed that CDYL2 mRNA is upregulated in four breast cancer cohorts within The Cancer Genome Atlas (TCGA) 34 (FIGS. 1A, 8A). Similarly, the NCBI GEO datasets GSE10780³⁵ and GSE21422³⁶ identified CDYL2 upregulation in invasive ductal breast carcinomas as well as ductal carcinoma in situ (DCIS), compared to normal breast tissues (FIG. 1A). Analysis of the paired Clinical Proteomic Tumor Analysis Consortium (CPTAC)³⁷ and TCGA datasets revealed that CDYL2 protein expression correlated with mRNA levels (FIG. 1B). We next asked if the expression level of CDYL2 correlates with clinical outcome. Patients were sub-divided into three categories based on their expression of the ER, progesterone receptor (PR) and HER2, namely ER+/HER2−, ER+/HER2+, and receptor triple-negative (TN). We found that high expression of CDYL2 correlated with worse survival in both ER+/HER2− and TN subtypes (FIG. 1C, D). Extending this analysis to other cancer contexts revealed an association between CDYL2 expression level and survival in colorectal carcinoma, rectal adenocarcinoma, lung squamous cell carcinoma, and lung adenocarcinoma (FIG. 8B-E). These findings identify CDYL2 as gene commonly upregulated in breast cancer and a candidate modulator of survival.

CDYL2 Over-Expression in the Non-Invasive Breast Cancer Cell Line MCF7 Induces Transcriptional Changes Associated with Malignant Progression

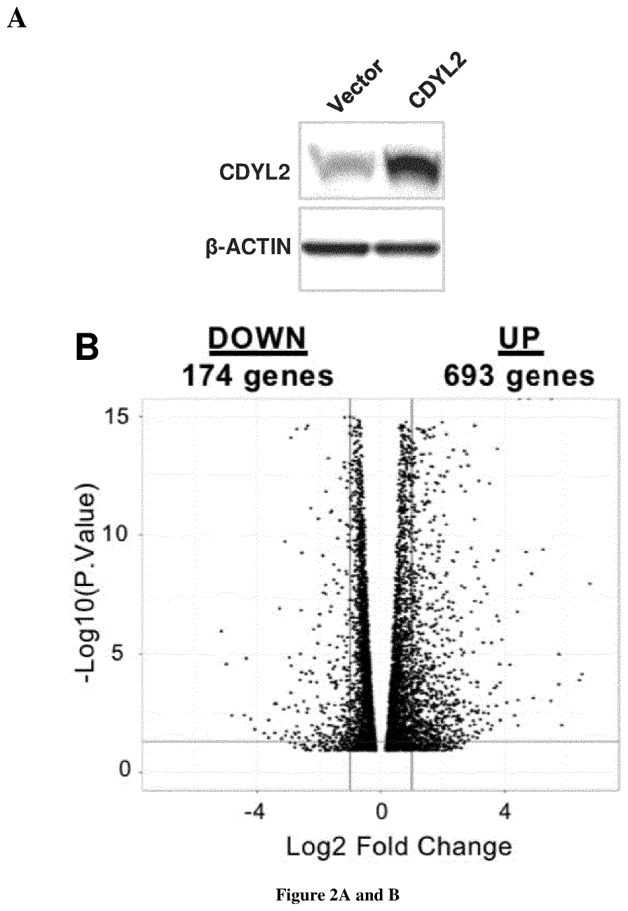

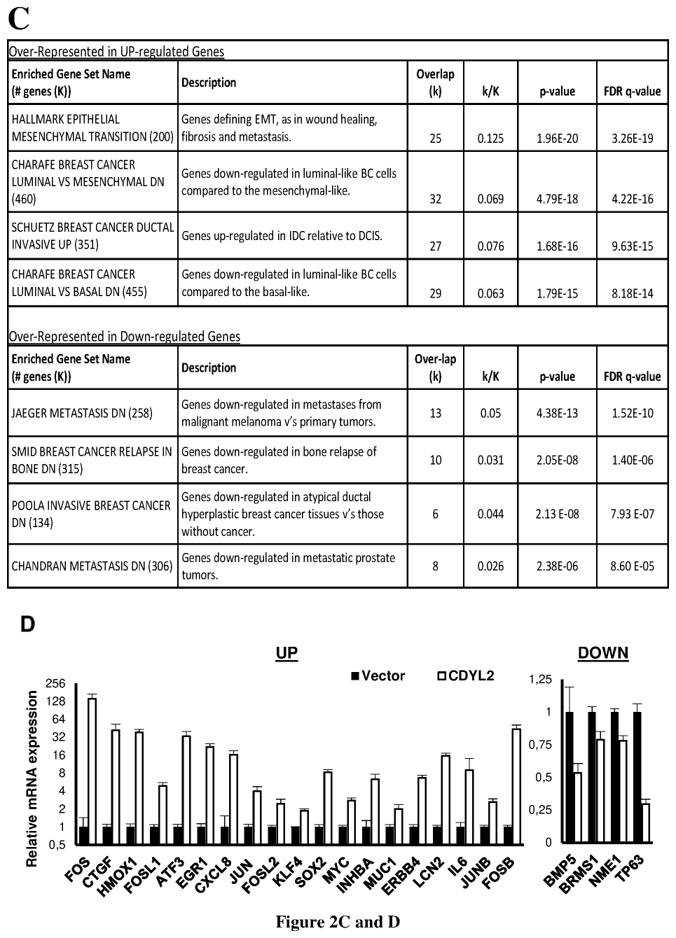

To ask if CDYL2 upregulation could induce oncogenic transcriptional and cellular changes, we stably expressed a CDYL2 cDNA in the non-invasive breast cancer cell line MCF7 (MCF7-CDYL2), or empty vector (MCF7-Vector) (FIG. 2A). While CDYL2 over-expression did not affect cell growth (Data not shown), RNA sequencing (RNA-Seq) revealed striking differences between MCF7-CDYL2 and MCF7-Vector cells, with 693 genes up-regulated and 174 genes down-regulated at least 2.5-fold (FIG. 2B). Gene set enrichment analysis (GSEA) of genes up- or down-regulated in MCF7-CDYL2 cells revealed positive associations with EMT, metastasis, invasive versus non-invasive ductal carcinoma, breast cancer relapse in bone, and atypical ductal hyperplasia compared to non-cancerous breast tissue (FIG. 2C). A number of genes from these GSEA signatures were validated by qRT-PCR, focusing on genes that are individually associated with breast cancer cell plasticity and malignant progression. These include the proto-oncogenes SOX2, KLF4, MYC, MUC1, FOS, FOSL1/Fra-1, and JUN³⁸⁻⁴⁴, and the secretory molecules LCN2, CTGF, CXCL8, INHBA and IL6^{45-47,47,48} (FIG. 2D). Down-regulation of the tumor suppressor TP63, breast metastasis suppressor BRMS1 and cytokine BMP5, which regulate EMT, metastasis, and stemness, among other processes^{49,50}, was also confirmed (FIG. 2D). Together, these insights suggest that CDYL2 over-expression can induce transcriptional changes associated with malignant breast cancer, potentially by promoting EMT, invasiveness and metastasis.

### CDYL2 Over-Expression in MCF7 Cells Induces EMT-Like Changes, Migration, Invasiveness and Mammosphere Formation

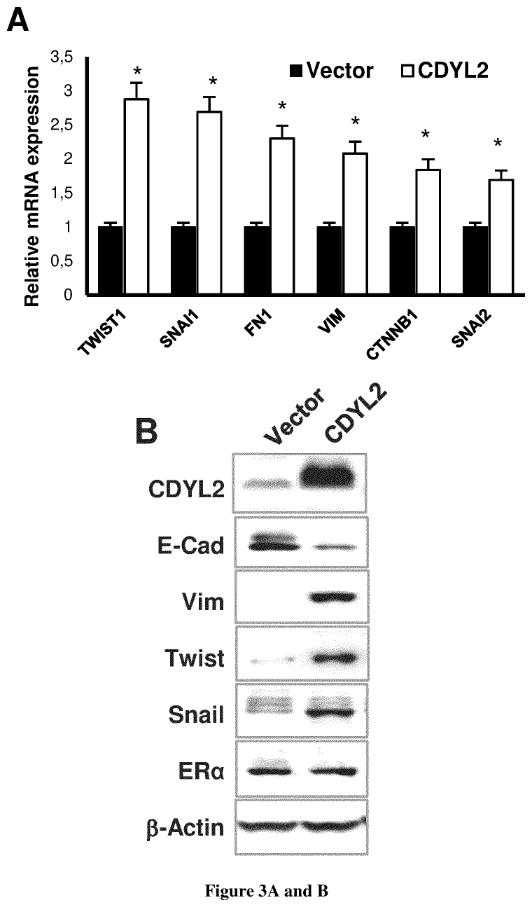

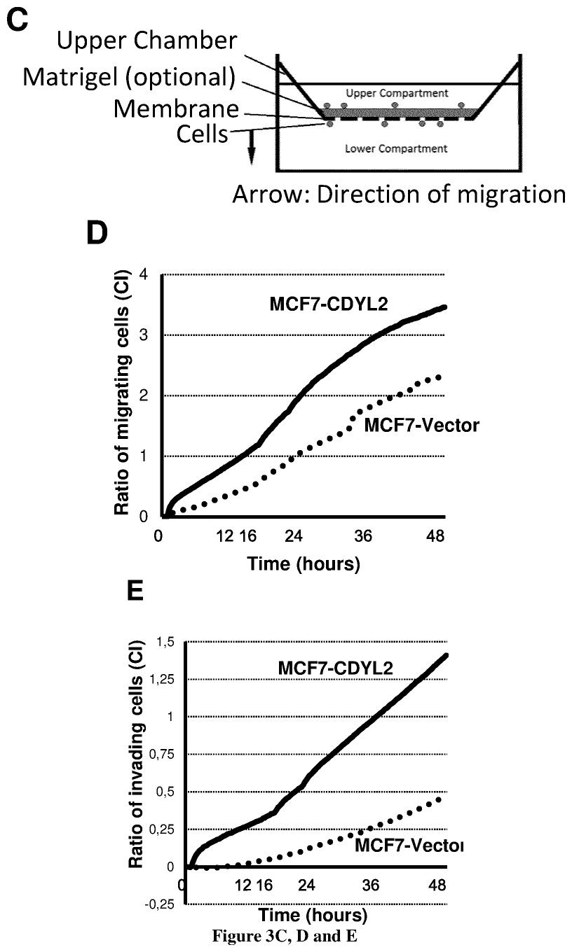

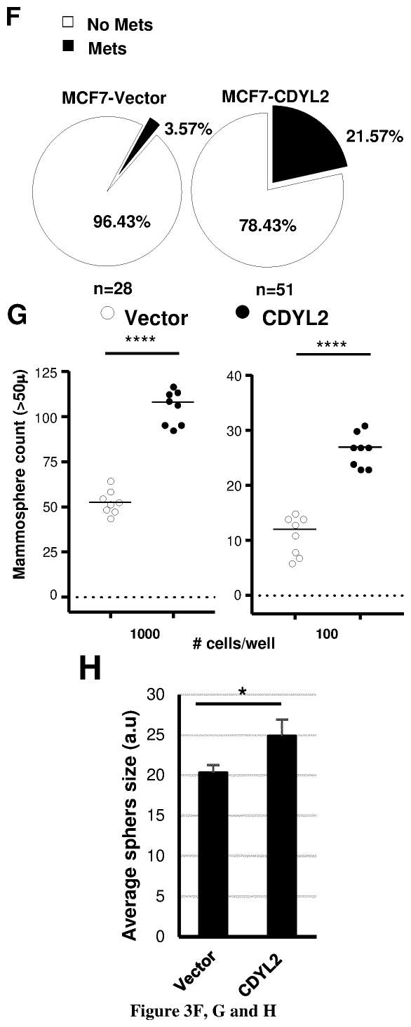

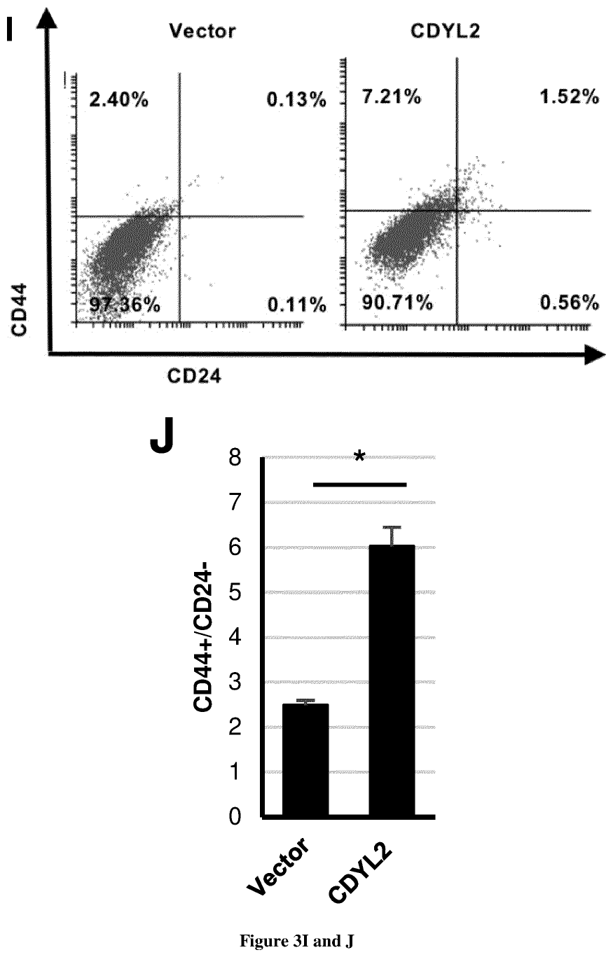

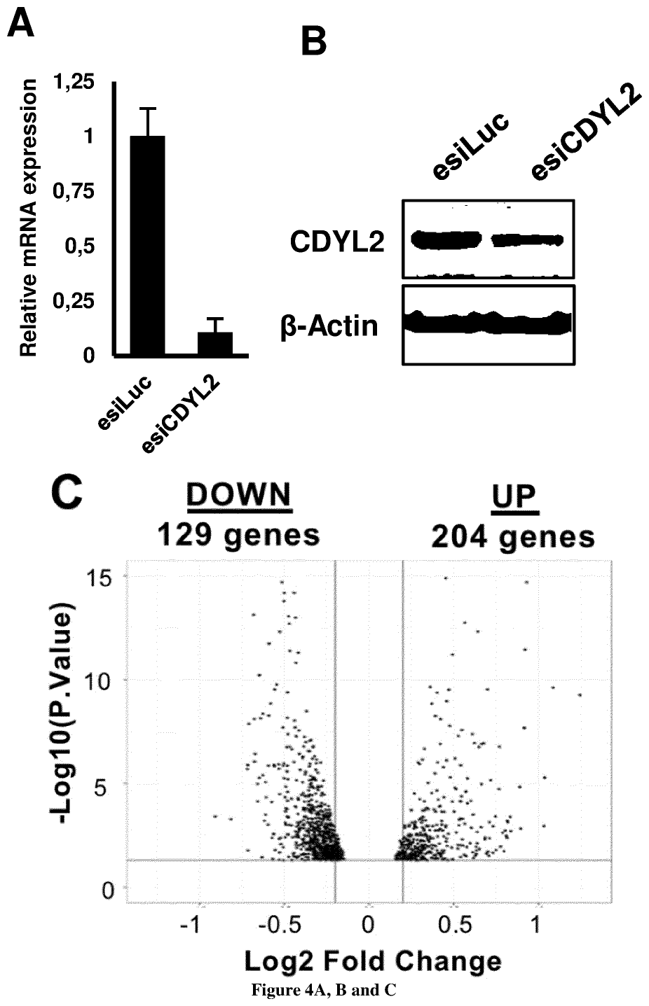

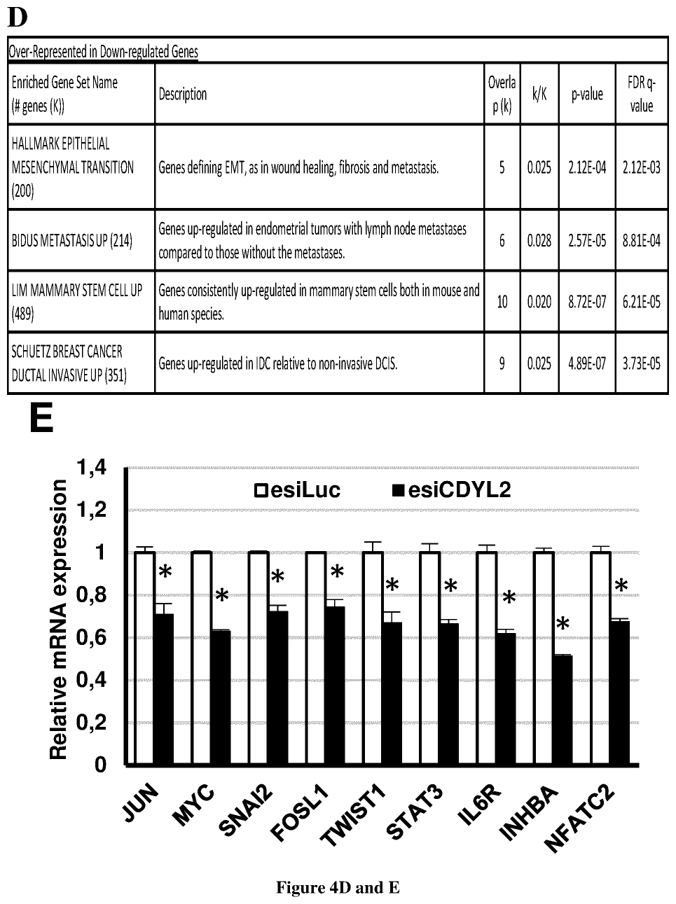

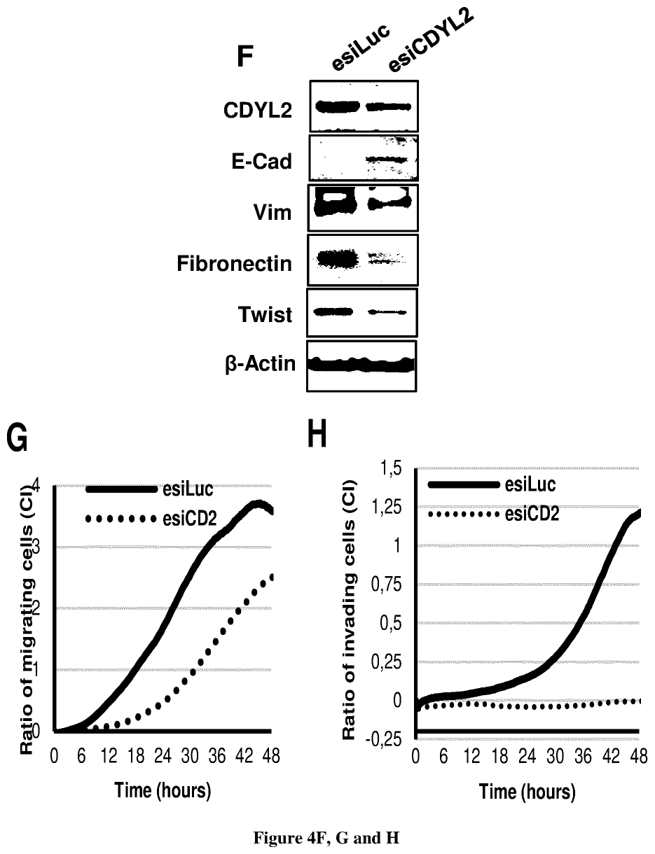

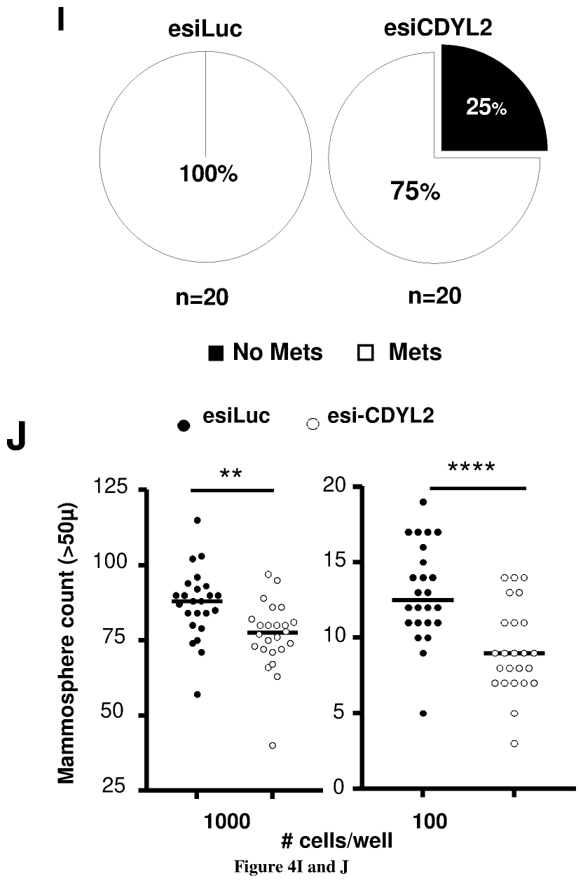

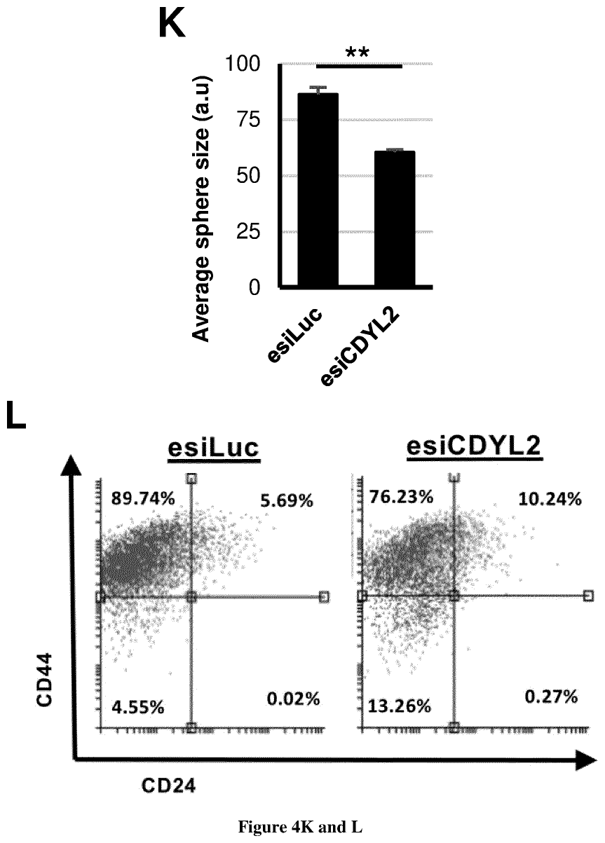

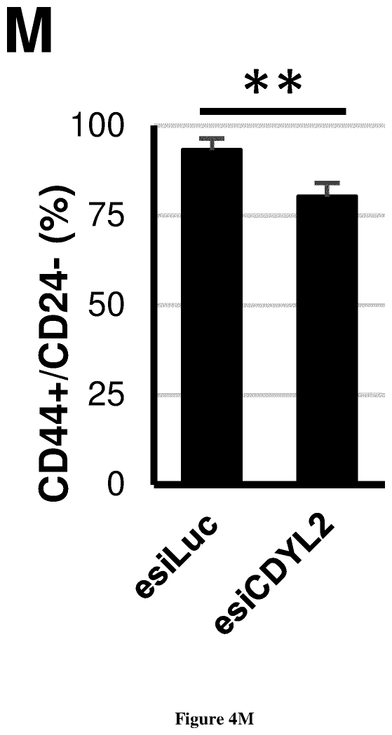

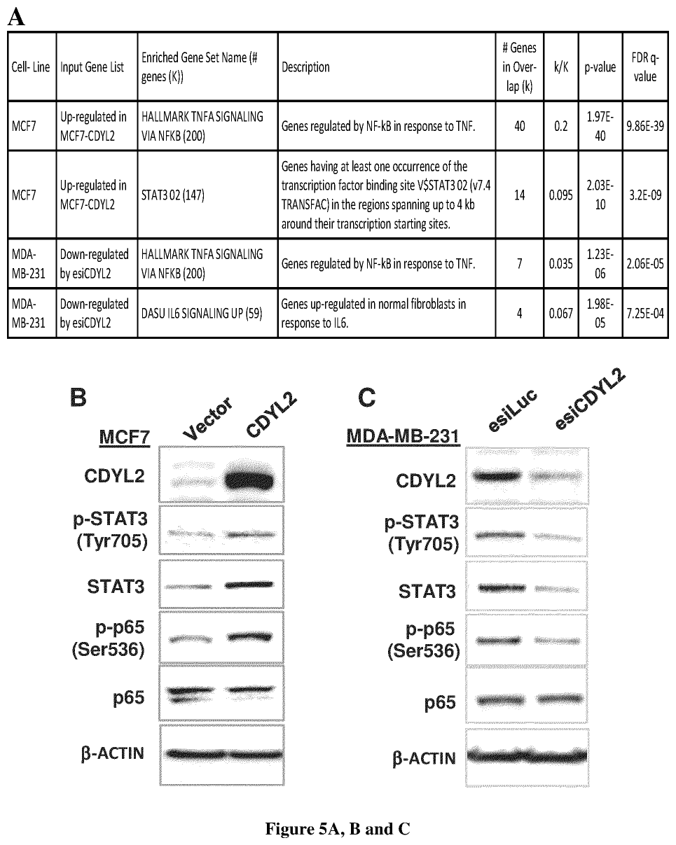

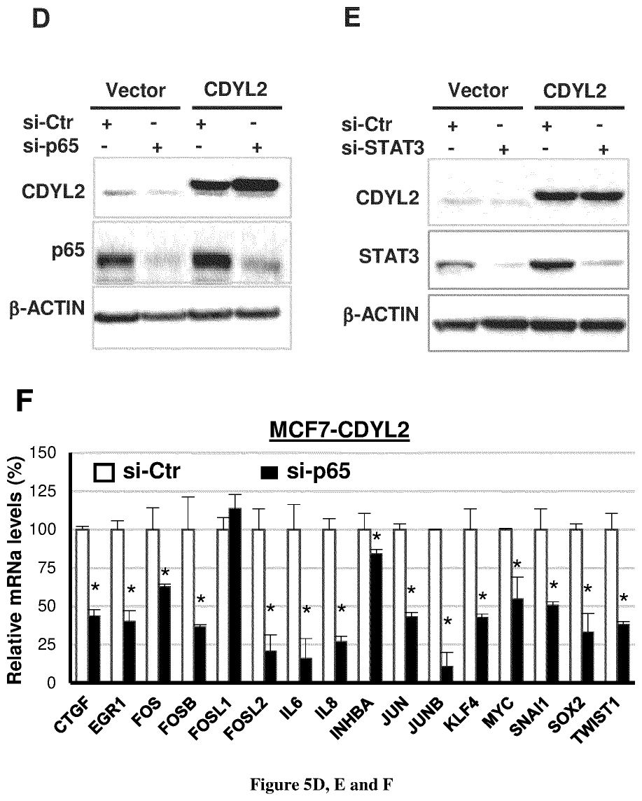

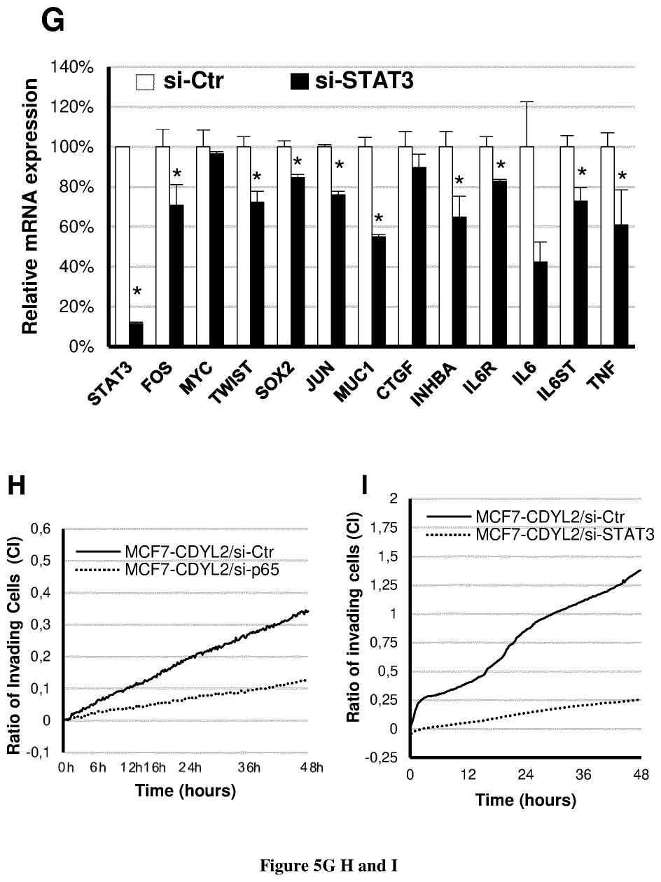

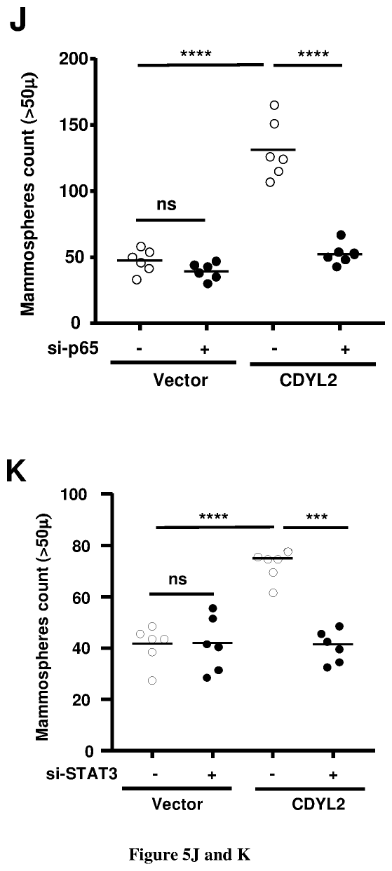

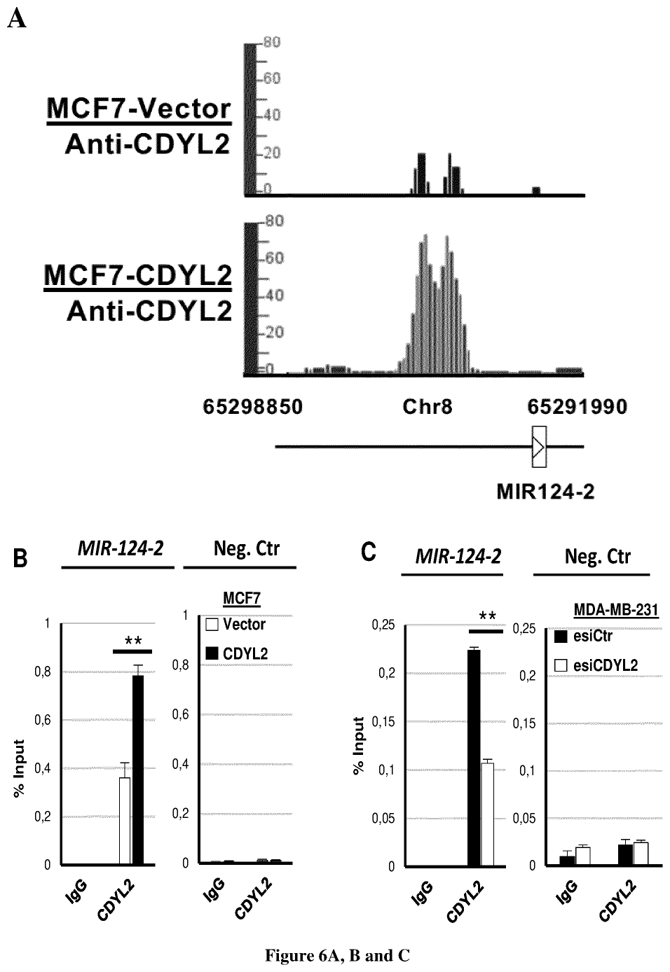

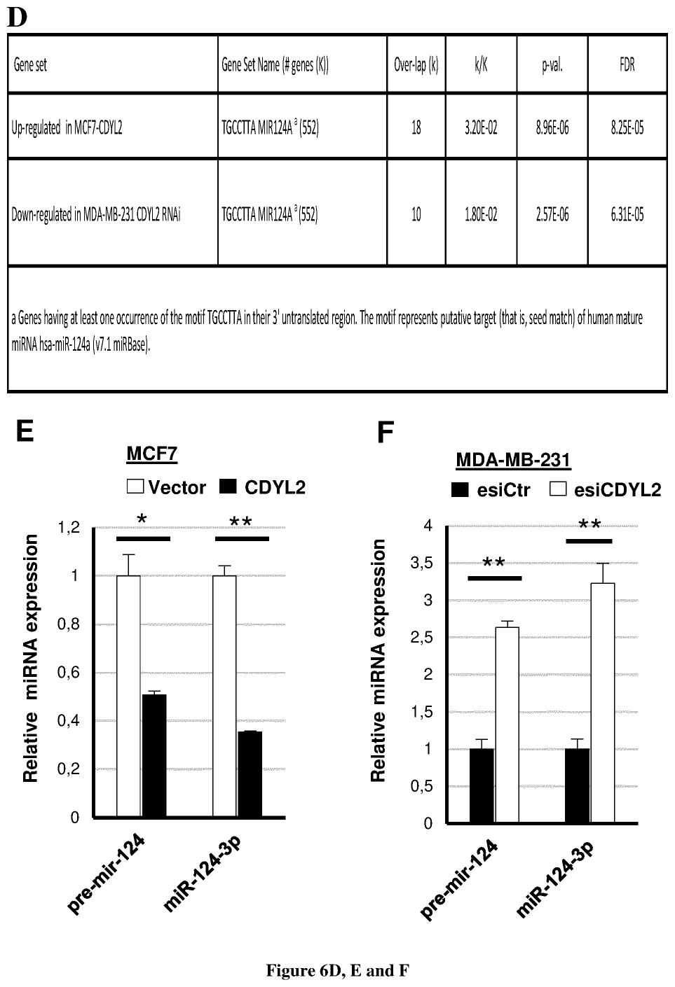

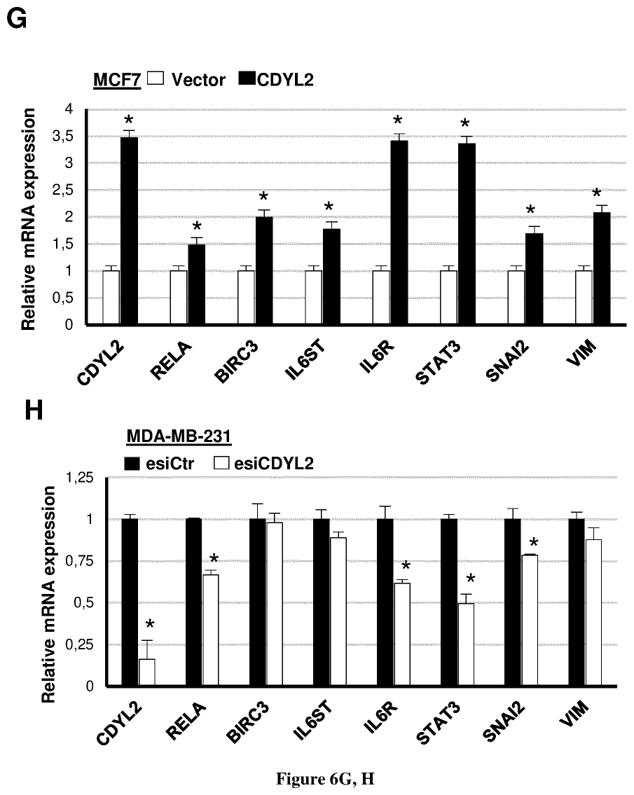

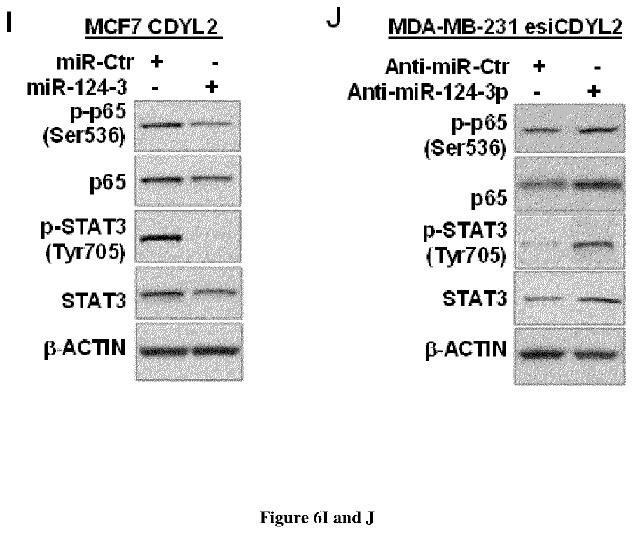

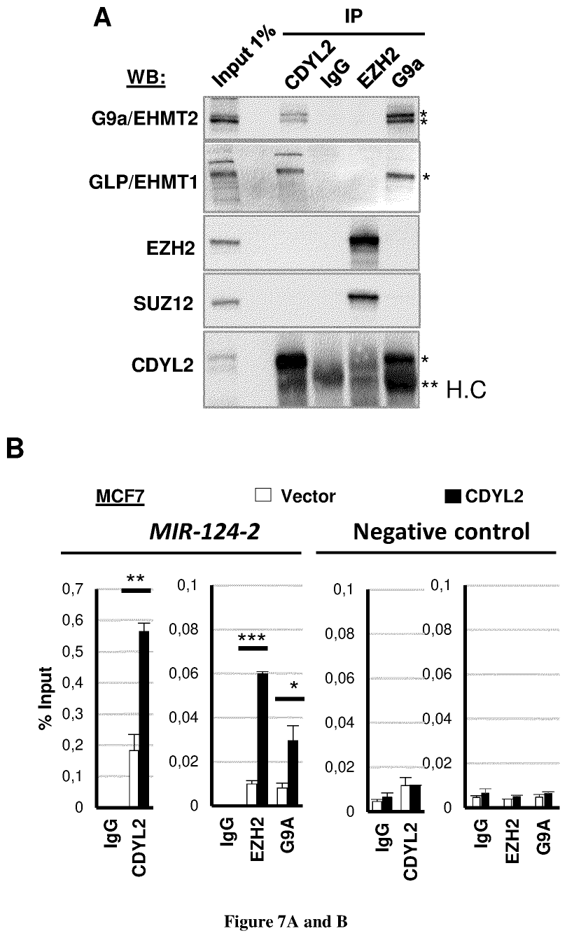

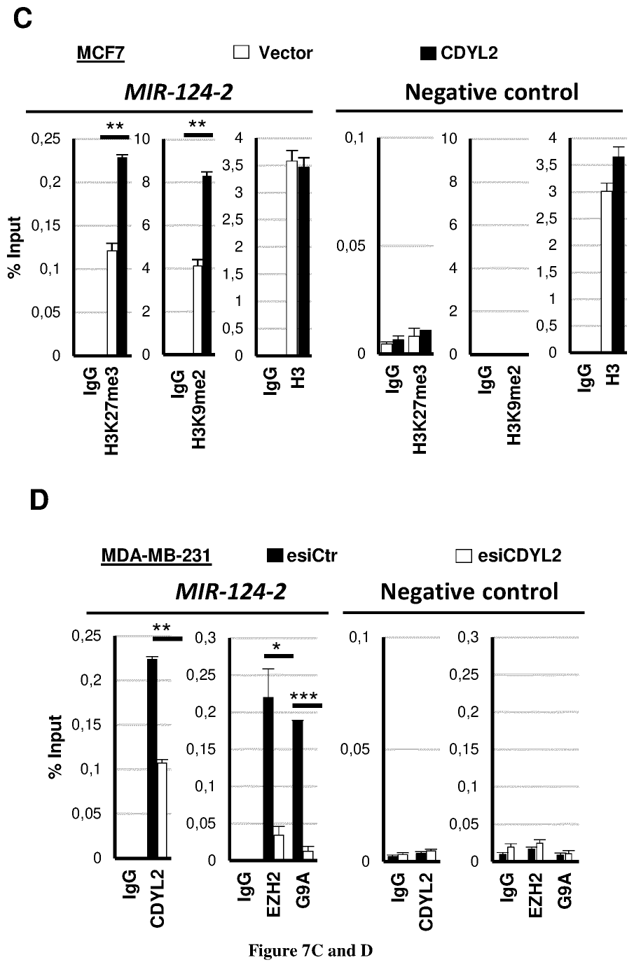

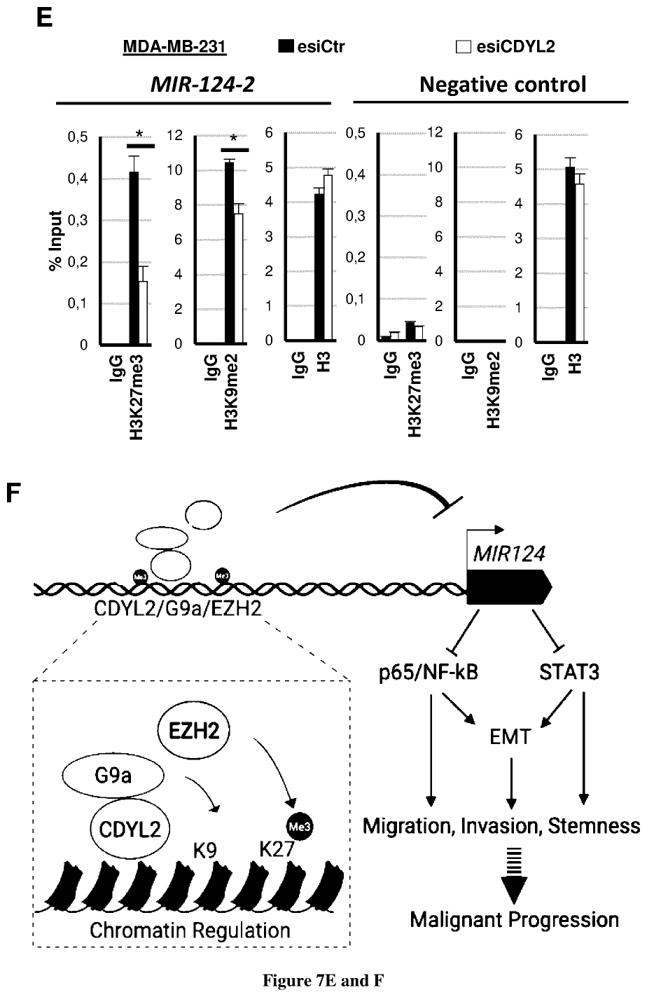

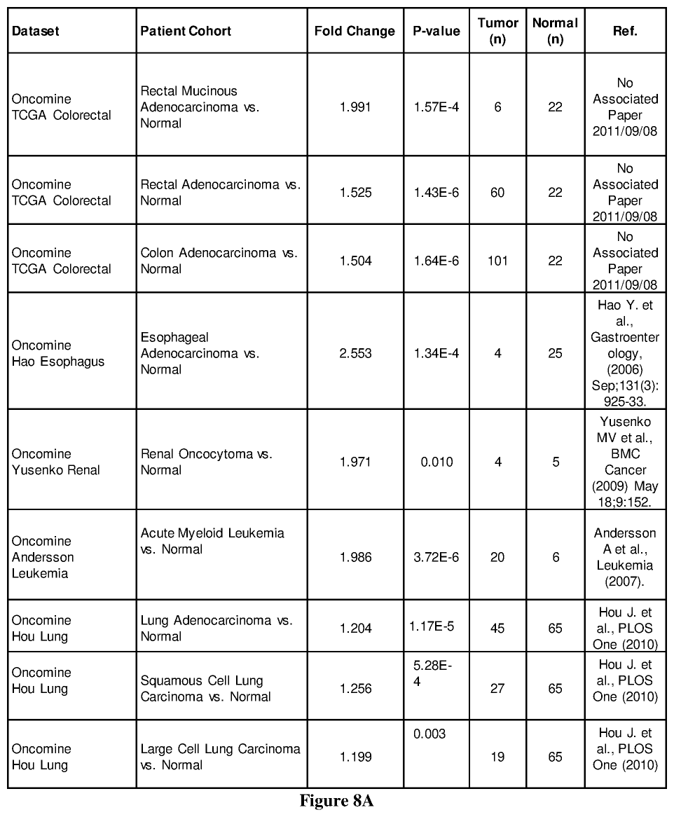

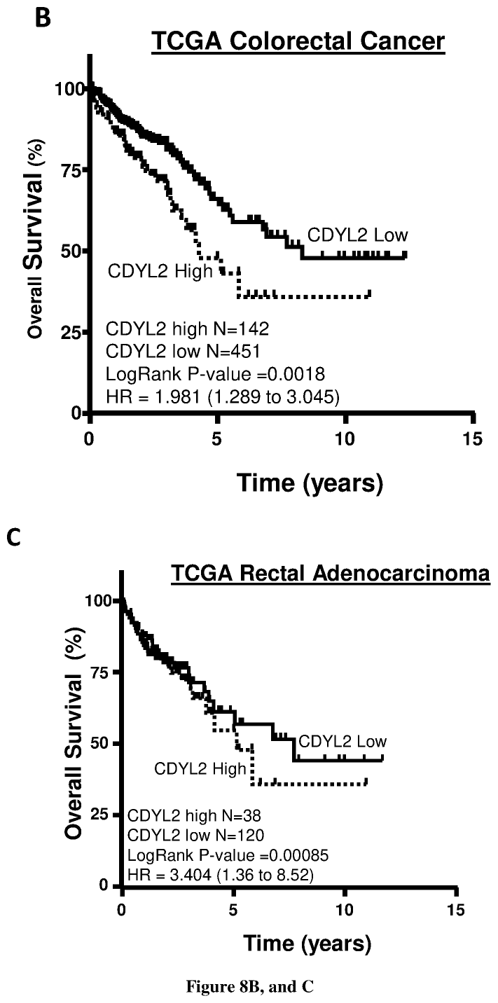

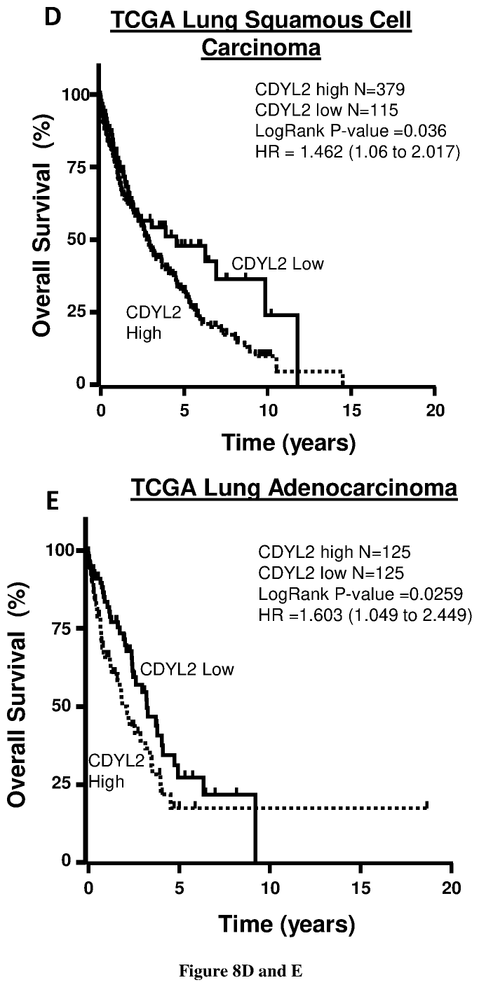

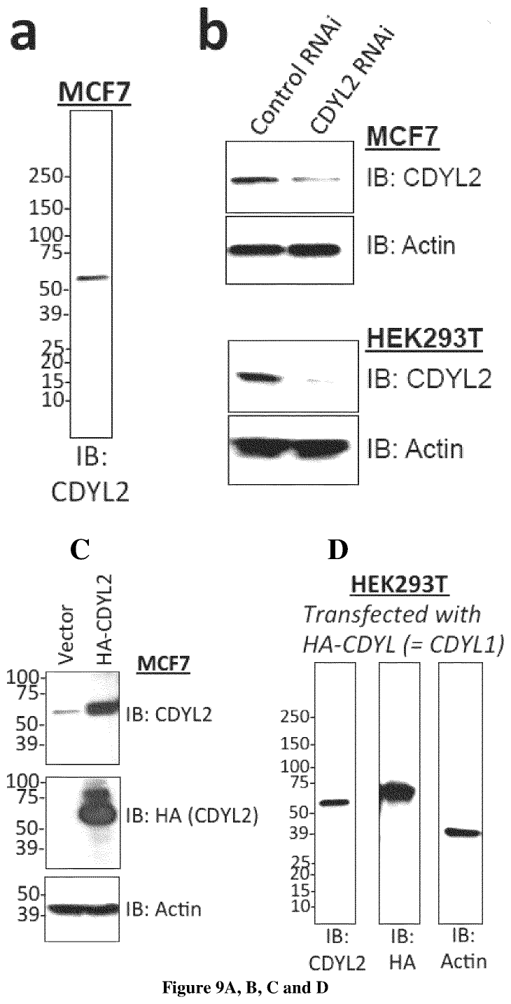

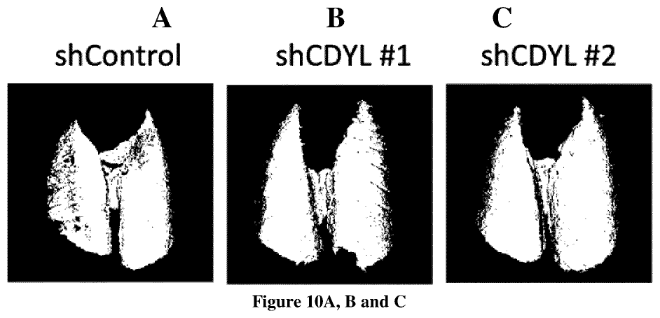

Further probing if CDYL2 might induce EMT-like changes in MCF7 cells, we assessed the expression of a panel of established EMT markers. qRT-PCR analysis revealed CDYL2 upregulation of mesenchymal markers TWIST1, SNAI1, FN1, VIM, CTNNB1 and SNAI2 (FIG. 3A). Western blotting revealed down-regulation of epithelial marker E-Cadherin and upregulation the mesenchymal markers Vimentin (VIM), TWIST1/Twist and SNAI1/Snail (FIG. 3B). However, CDYL2 over-expression did not alter the levels of ER-alpha (FIG. 3B), downregulation of which can induce EMT⁵¹, suggesting an independent mechanism. Notably, three weeks after MCF7 cells were transduced with the CDYL2 over-expression construct a change in cell morphology occurred, with loss of the cobblestone-like morphology of monolayers, replaced by a more fibroblast-like morphology, similar to previous descriptions of EMT in MCF7^{52,53}.

Among the primary contributions of EMT to malignant progression is increased cancer cell migration and invasion. In vitro assays revealed that the MCF7-CDYL2 cells migrated more proficiently across a microporous membrane compared to controls (FIG. 3C,D). Using an adaptation of this assay to test for invasive capacity, wherein the porous membrane was first overlaid with a Matrigel barrier, we found that MCF7-CDYL2 cells also had increased invasive capacity relative to controls (FIG. 3E).

We then probed the effect of CDYL2 on MCF7 cell invasion and metastasis in vivo. Both MCF7-CDYL2 and control cells were fluorescently labeled and injected into the perivitelline space of zebrafish embryos. The presence of tail metastases was monitored by fluorescence microscopy 24 h later. Whereas MCF7-Vector cells rarely produced metastases (3.57% of fish), MCF7-CDYL2 cells did so in 21.57% of cases (FIG. 3F).

To test if CDYL2 overexpression additionally induced stem-like characteristics in MCF7 cells, we first performed a mammosphere assay. This is a functional assay to assess the enrichment of stem-like cells in a population. MCF7-CDYL2 cells yielded both more and larger mammospheres compared to controls (FIG. 3G-H). Consistent with this, flow cytometry analysis revealed that CDYL2 over-expression in MCF7 cells increased the fraction of cells bearing the stem-like antigenic profile of CD44-high/CD24-low cells compared to controls (FIG. 31, J). Taken together, this series of studies indicate that CDYL2 over-expression in MCF7 cells promotes a number of cellular phenotypes associated with cellular plasticity and malignant progression.

### RNAi Knockdown of CDYL2 in the Invasive Breast Cancer Cell Line MDA-MB-231 Diminishes the Expression of EMT Markers and Inhibits Migration, Invasion, and Mammosphere Formation

We next analyzed the effect of CDYL2 loss of function in the highly invasive, cancer stem cell enriched, mesenchymal-like breast cancer line MDA-MB-231. CDYL2 expression was inhibited by RNAi (FIGS. 4A, B) and RNA-seq performed. Compared to CDYL2 over-expression in MCF7 cells, the effects of its knock-down on MDA-MB-231 cell gene expression were moderate, with no genes up- or down-regulated 2.5-fold or greater, except for CDYL2 itself (Data not shown). However, using a fold-change cut-off of 1.25, we identified 204 genes up-regulated and 129 genes down-regulated (FIG. 4C). GSEA analysis revealed that the down-regulated gene set was enriched in transcripts associated with EMT, metastasis, mammary stem cells and invasive ductal carcinoma (FIG. 4D). This suggests that CDYL2 knock-down might suppress EMT, metastasis, and stemness. To determine if this is the case, we performed essentially the same suite of assays used to probe the effect of CDYL2 over-expression on MCF7 cells (FIG. 3).

We first confirmed by qRT-PCR that CDYL2 RNAi reduced the levels of a number of transcripts associated with EMT, namely JUN, MYC, SNAI2, FOSL1 and TWIST1 (FIG. 4E). Immunoblotting revealed induction of E-cadherin expression, and diminished expression of Vimentin, Fibronectin, and Twist (FIG. 4F). We did not observe morphological changes in these cells indicative of a mesenchymal-to-epithelial transition (MET), possibly because the duration of the RNAi treatment was not long enough for such phenotypes to emerge. However, transduction of MDA-MB-231 cells with three independent shRNA sequences targeting CDYL2 (Data not shown) induced epithelial-like morphological changes after two weeks, with cells forming cobblestone-like monolayers (Data not shown). These cells also exhibited down-regulation of a number genes associated with EMT-induction that were strongly up-regulated in MCF7-CDYL2 cells (FOS, FOSB, JUNB, CXCL8, CTGF, LCN2, MUC1, ERBB4), but not down-regulated in MDA-MB-231 cells treated with transient CDYL2 RNAi (Data not shown). In vitro assays revealed that transient or stable RNAi of CDYL2 dramatically reduced the migratory and invasive ability of MDA-MB-231 cells, relative to controls (FIG. 4G, H). In vivo, CDYL2 RNAi significantly impaired the ability of MDA-MB-231 cells to metastasize from the perivitelline site of injection to the tail of zebrafish embryos (FIG. 4I), indicating suppression of the invasive and/or migratory capacity. Knockdown of CDYL2 by transient or stable RNAi treatment also resulted in fewer and smaller mammospheres relative to negative controls (FIG. 4J-K). While FACS analysis revealed that the majority of control RNAi-treated MDA-MB-231 cells were CD44-high/CD24-low, CDYL2 RNAi induced a population of CD44-low/CD24-low cells, indicative of loss of stemness (FIG. 4L, M). Collectively, these assays indicate that CDYL2 is required for MDA-MB-231 cell migration, invasion and stemness, as well as the full expression of its mesenchymal-like state.

Regulation of p65/NF-κB and STAT3 Signaling by CDYL2

GSEA analysis of the effects of CDYL2 over-expression in MCF7 cells or knockdown in MDA-MB-231 revealed a potential role in regulating NF-κB/TNF-alpha and STAT3/Interleukin-6 signaling (FIG. 5A). We asked if the regulation of these pathways by CDYL2 might contribute to its regulation of EMT, invasion and stemness. Accordingly, over-expression of CDYL2 in MCF7 cells increased the levels of tyrosine 705-phosphorylated STAT3 (FIG. 5B), the active form of this protein. It also increased the levels of serine 536-phosphorylation on the NF-cB TF p65 (FIG. 5B), indicating an increase in canonical NF-cB pathway signaling. By contrast, the levels of both phosphoproteins were diminished in CDYL2 RNAi treated MDA-MB-231 cells (FIG. 5C). In addition, while the total p65 levels were not affected by either CDYL2 over-expression or RNAi, total STAT3 levels were higher in MCF7-CDYL2 cells compared to controls (FIG. 5B) and downregulated after CDYL2 RNAi in MDA-MB-231 cells (FIG. 5C).

We then asked if CDYL2 induction of genes associated with EMT, invasion, and sternness in MCF7 cells might be dependent on signaling via p65/NF-κB and STAT3. qRT-PCR analysis revealed that p65 RNAi downregulated several genes associated with NF-κB signaling, namely CTGF, EGR1, FOS, IL6, CXCL8, INHBA, JUN, MYC, SNAI1, KLF4, SOX2 and TWIST1 (FIG. 5D,F). Similarly, STAT3 RNAi downregulated several genes associated with STAT3 signaling, including FOS, TWIST1, SOX2, JUN, MUC1, INHBA, IL6R, IL6ST, and TNF (FIG. 5E,G). Strikingly, RNAi knockdown of either p65 or STAT3 potently suppressed both invasiveness (FIG. 5H, I) and mammosphere induction by CDYL2 (FIG. 5J, K). These analyses indicate that p65/NF-κB and STAT3 signaling is regulated by CDYL2, and both pathways are required for CDYL2 induction of invasion and mammosphere formation in MCF7 cells.

CDYL2 binds upstream of MIR124-2 gene and regulates miR-124 expression Consistent with the possibility that CDYL2 might be an epigenetic regulator of transcription, we found that it was enriched in the nucleus of both MCF7 and MDA-MB-231 cells, with a significant fraction present in the chromatin fraction (Data not shown). To identify where on chromatin CDYL2 is bound, we performed CDYL2 Chromatin Immunoprecipitation in both MCF7-Vector and MCF7-CDYL2 cells followed by Illumina sequencing (ChIP-seq). This revealed several genomic loci that were more enriched in CDYL2 in the MCF7-CDYL2 cells compared to vector controls, including upstream of all three members of the MIR124 gene family (FIG. 6A). We confirmed this observation using ChIP-qPCR (FIG. 6B). A non-reactive IgG was used as negative ChIP control, while qPCR analysis did not detect enrichment of CDYL2 at an unrelated sequence (FIG. 6B). We reasoned that CDYL2 repression of MIR124 genes might contribute to its regulation of STAT3 and NF-κB signaling in MCF7 and MDA-MB-231 cells. In agreement with this possibility, CDYL2 RNAi diminished its levels upstream of MIR124 genes in MDA-MB-231 (FIG. 6C), with a corresponding increase in the expression of both the precursor (pre-mir-124) and mature (miR-124-3p) forms of microRNA-124 (FIG. 6F). Stable knock-down of CDYL2 using three independent shRNAs also increased levels of both pre-mir-124 and miR-124-3p (data not shown). In complementary analysis, both MIR124 transcripts were downregulated by CDYL2 over-expression in MCF7 (FIG. 6E). Supporting the notion that the alterations of miR-124 levels were sufficient to affect cell function, analysis of the MCF7-CDYL2 and MDA-MB-231 esiCDYL2 RNA-seq data showed that miR-124 target genes were commonly upregulated in the former, and down-regulated in the latter (FIG. 6D). Differential expression of several of these genes was validated by RT-qPCR (FIG. 6G,H). In suppression assays, a miR-124-3p mimic strongly diminished the levels of the active, phosphorylated forms of both p65 and STAT3 (FIG. 6I). miR-124-3p also suppressed the total levels of STAT3 protein (FIG. 6I). In complementary experiments, a neutralizing anti-miR-124-3p oligonucleotide rescued esiCDYL2 suppression of phospho-p65 and phospho-STAT3 levels in MDA-MB-231 cells, compared to a control non-targeting anti-miR oligonucleotide (FIG. 6J). The reduced total STAT3 levels observed upon esiCDYL2 treatment were also rescued by anti-miR-124-3p treatment (FIG. 6J). These findings indicate that CDYL2 regulates miR-124 levels, possibly via its binding upstream of MIR124 genes, and that control of miR-124-3p levels by CDYL2 contributes to its regulation of NF-κB and STAT3 signaling.

CDYL2 Interacts with G9a, GLP, and PRC2 Complex Components EZH2 and SUZ12

Because CDYL2 is enriched at MIR124 genes and negatively regulates miR-124 expression, we asked if it might promote an epigenetically repressive chromatin environment at these loci. However, the epigenetic mechanism of CDYL2 is not known. By analogy with CDYL1, we speculated that it may form a complex with the H3K9 di-methyltransferases G9a, GLP or SETDB1²⁵ and the Polycomb Repressive Complex 2 (PRC2) core components EZH2 and SUZ12²⁷. Using immunoprecipitation (IP) assays we found that Anti-CDYL2, but not a control IgG, efficiently recovered endogenous CDYL2 from MCF7 lysates and co-immunoprecipitated (CoIP) G9a and GLP (FIG. 7A). We also detected the presence of small amounts of EZH2 and SUZ12 in the CDYL2 IP (data not shown, but at a much lower percentage of input compared to G9a and GLP, suggesting a low-abundance or labile interaction. Reciprocal CoIP assays confirmed G9a interaction with CDYL2, but did not identify CDYL2 association with EZH2 (FIG. 7A). These data indicate that CDYL2 forms a complex with G9a and GLP, and may interact marginally with EZH2 and SUZ12.

CDYL2 Regulates the Enrichment of G9a and EZH2 Upstream of MIR124 Genes, as Well as that of their Cognate Methylation Marks H3K9Me2 and H3K27Me3

We next asked if CDYL2 might control the levels of G9a and EZH2 at a promoter-proximal region upstream of MIR124 genes. ChIP-qPCR assays indicated that CDYL2, G9a and EZH2 were enriched upstream of these genes in both MCF7 and MDA-MB-231 cells (FIG. 7B, C). The enrichment of both methyltransferases was increased by CDYL2 over-expression in MCF7 (FIG. 7B) and diminished by CDYL2 RNAi knockdown in MDA-MB-231 (FIG. 7D). Increased levels of H3K9me2 and H3K27me3 were also observed upstream of MIR124 genes in MCF7-CDYL2 (FIG. 7C), while levels of H3K9me2 and H3K27me3 at these loci were decreased upon CDYL2 RNAi in MDA-MB-231 (FIG. 7E). The levels of total histone H3 at these loci were not affected by CDYL2 over-expression in MCF7 or its knockdown in MDA-MB-231 (FIG. 7C, E, left panels). The same pattern of alterations was not observed at two independent control sequences (FIG. 7C, E, right panels). These findings indicate that in addition to interacting with G9a, and weakly so with EZH2, CDYL2 positively regulates the enrichment of both methyltransferases upstream of MIR124 genes, as well as those of the histone marks they regulate.

### Discussion

Despite the emergence of epigenetic factors as important regulators of cancer cell plasticity and malignant progression, the underlying molecular mechanisms remain poorly understood. This is due in part to insufficient characterization of several putative epigenetic factors, including CDYL2. Our study shows that CDYL2 is frequently misexpressed in breast cancer, and provides a proof-of-principle that this could promote cellular phenotypes associated with malignant progression. We present the first insights into the genes and cellular pathways CDYL2 controls, and the epigenetic mechanisms it engages. Based on our findings, we propose that CDYL2 upregulation contributes to poor prognosis in breast cancer by inducing epigenetic deregulation of genes and pathways important in tumorigenesis (MIR124, NF-κB, STAT3), resulting in cellular changes central to malignant progression (EMT, migration, invasion, stemness).

Although we predicted CDYL2 to be an epigenetic repressor of transcription due to its homology to CDYL1^{22,23}, this was not previously demonstrated. Our data support a mechanism whereby CDYL2 regulates the levels of G9a and EZH2 and their cognate histone methyl-lysine marks upstream of MIR124 genes, creating a local epigenetic environment repressive to transcription (schematic diagram, FIG. 7F). While our CoIP data suggest that CDYL2 might regulate G9a levels at MIR124 genes via a mechanism involving physical association of the two factors, they only weakly support this possibility in the case of EZH2. We speculate that an indirect mechanism could account for the strong effects of CDYL2 over-expression and RNAi on EZH2 enrichment upstream of MIR124 genes, such as the previously described regulation of EZH2 levels on chromatin by G9a methyltransferase activity⁵⁴.

MIR124 genes are emerging tumor suppressors commonly silenced in various cancers including breast^{15,20,48,55}. MiR-124-3p directly targets STAT3 mRNA and antagonizes p65/NF-κB by inhibiting multiple components of its signaling pathway. It also regulates EMT, migration, invasion, and stemness¹³⁻¹⁵ Importantly, we show that CDYL2 positively regulated the active forms of both STAT3 and p65 in a manner reliant on miR-124 levels. We conclude that CDYL2 regulation of miR-124 expression substantially accounts for CDYL2 regulation of p65/NF-κB and STAT3 signaling, though we cannot exclude the possibility that other factors also contribute.

Both STAT3 and p65/NF-κB signaling are known drivers of cancer cell plasticity and malignant progression 3-5,7,15,16,18 In addition to positively regulating these pathways, CDYL2 also induced several cellular phenotypes associated with plasticity and aggressiveness in breast cancer, namely increased migration, invasiveness and stem-like behavior. Significantly, the ability of CDYL2 to induce MCF7 cell invasion and mammosphere formation was suppressed by inhibition of either p65/NF-κB or STAT3, indicating a crucial role for these pathways in its putative oncogenic mechanism.

It has been proposed that in certain malignancies, including breast cancer, molecular and cellular changes that promote the emergence of mesenchymal-like cells constitute a key enabling step in the process of malignant progression^{56 59}. The EMT paradigm now encompasses a diversity of molecular and cellular expressions, several of which were positively regulated by CDYL2. These include changes in established EMT markers, as well as in cell morphology, migration, invasion and stemness. Notably, as was the case for invasion and stemness, CDYL2 induction of an EMT-like gene expression program in MCF7 cells was partially reversed by inhibition of STAT3 or p65, indicating it is downstream of these pathways. Overall, our studies are consistent with an oncogenic effect of CDYL2 over-expression in breast cancer. This might contribute to the poor prognosis of the ER+/HER2− and TN breast cancer patients whose cancers express high levels of CDYL2. Although not studied in depth here, we also observed a correlation between high CDYL2 expression and poor prognosis in lung and colorectal carcinomas, hinting at a wider role in cancer. Given the emergence of epigenetic factors as viable therapeutic targets in cancer, our study supports the further evaluation of CDYL2 as a candidate drug target in breast cancer, and potentially other malignancies. Notably, molecular and cellular changes associated with EMT and stemness in cancer cells were proposed to underlie resistance to a range of cancer therapies, as well as increased propensity to form invasive and metastatic tumors (42-44; 57, 59). Therefore, CDYL2 inhibition may also be effective in treating therapy resistant or malignant cancers.

### EXAMPLE 2: CDYL2 INHIBITION AS A STRATEGY TO INCREASE TUMOR CELL IMMUNOGENICITY AND REDUCE CANCER IMMUNE EVASION

### Materials and Methods:

### Cell Culture

MCF7 cells (ATCC, HTB-22) and their derivatives were grown in DMEM Low Glucose (Gibco, 31885-023) supplemented with 10% of FBS (Gibco, 10270-106), 40 μg/mL of gentamicin (Gibco, 15710-049) and 0.6 μg/mL of insulin (NovoRapid, 3525909). MDA-MB-231 cells (ATCC, HTB-26) were grown in DMEM GlutaMAX (Gibco, 10566016) supplemented with 10% of FBS (Gibco, 10270-106), 1% penicillin/streptomycin (Gibco, 15140122). Cells were grown at 37° C. and 5% C02 in a humidified incubator and passaged every 2-4 days by trypsinization. Sustained expression of ER-alpha in MCF7 was validated regularly by western blotting and immunofluorescence. Cells were regularly tested for mycoplasma using a commercial kit (ATCC, 30-1012K), and cultures renewed from low passage stocks every two months or less.

### Stable Expression of CDYL2 in MCF7 Cells

CDYL2 cDNA was cloned by PCR from an MCF7 cDNA library using the primers in Table S3 and Phusion polymerase (NEB, M0530), and inserted into the Gateway pENTR-D-TOPO vector (Invitrogen, K240020). Sequencing on both strands confirmed that the cDNA corresponded to a published CDYL2 sequence (Genbank, NM_152342.2). The cloned cDNA was then transferred into MSCV plasmid (Addgene #41033) using LR Clonase (Invitrogen, 11791100), and the resulting expression construct validated by sequencing. MCF7 were then stably transduced with MSCV (Vector) or MSCV-CDYL2 retroviruses and selected for 14 days using 2 μg/mL puromycin (Sigma, P8833). Expression was confirmed by western blotting and immunofluorescence using CDYL2 antibody.

### RNA Interference

MCF-7 or MDA-MB-231 cells were transfected with CDYL2 esiRNA (Sigma, EHU042511) or esiLuciferase (Sigma, EHUFLUC) using Interferin reagent (Polyplus, 409-10) according to the manufacturer's instructions. Cellular assays and analysis were performed between 48 and 72 h post-transfection, according to the experiment.

### RNA-Seq and Enrichment Analysis

RNA was extracted using TRI-reagent (Sigma, T9424) and libraries were prepared with the TruSeq Stranded Total-RNA kit and sequenced on a Illumina NextSeq sequencing machine. After careful quality controls, raw data were aligned on the human genome (hg38) with STAR v2.7.0f (Dobin et al., (2013) Bioinformatics) and default parameters. Read counts on each genes of the Gencode annotation v29 were produced by STAR. Unless otherwise specified, the analyses were performed using R (v 3.4.4) and illustrations produced with the ggplot2 (Wickham, H., (2016) Elegant Graphics for Data Analysis) and ggpubr packages. Starting from raw counts, we used the R package DESeq2 (v1.14) (Love et al, Genome Biology, 2014) to perform the differential expression analyses. Each time the design was set as ˜REP+TYPE, where REP refers to the replicate number (paired analysis) and TYPE to the treatment group. Differential expression was tested using the Wald test, and p-values were corrected with the Benjamini-Hochberg method. GSEA analysis was performed as described (Subramanian et al., (2005) Proc. Natl. Acad. Sci. U.S.A.), using the MSigDB collection of gene signatures (Liberzon et al., (2011) Bioinformatics).

### Anti-CDYL2 Production:

CDYL2 cDNA was cloned from MCF-7 cells into pENTR(D)-Topo plasmid (Invitrogen), then transferred using LR Clonase (Invitrogen) into an N-terminal 6-Histidine tagging bacterial expression plasmid (pET-28a(+), EMD Biosciences) that was previously adapted for use as an LR Clonase Destination plasmid using the gateway system (Invitrogen). Next we expressed this plasmid in DE3-pLysS E. coli (Promega), and purified the resulting soluble, non-denatured His6-CDYL2 using NiNTA agarose beads (Qiagen) essentially as described in the NiNTA bead manual. The resulting purified Hisr-CDYL2 was used to immunise two rabbits and generate polyclonal anti-CDYL2 sera (immunization conducted by Covalab, Lyon, France). The antisera thus produced were tested at various points along the immunisation protocol.

### Results and Discussion:

Both MCF-7 and MDA-MB-231 cell lines were treated with either siRNA targeting CDYL2 (esiCDYL2) or control siRNA targeting the firefly luciferase gene (esiLuc), and RNA extracted after 48 h in the case of MCF-7 and after 72 h in the case of MDA-MB-231 cells. The time of RNA harvesting was determined by doing a time course experiment to identify the point of maximum CDYL2 knock-down efficiency. In both cases, the experiment was repeated three times. The resulting triplicate samples were analyzed by paired-end (PE) total RNA sequencing (RNA-seq), and differences in gene expression between the esiCDYL2 and esiLuc groups determined. From the resulting data we derived a list of genes ranked from the most up-regulated in the esiCDYL2 dataset relative to esiLuc, to the most down-regulated. Comparison of the ranked gene lists to previously published gene expression signatures in the Molecular Signatures Database (MSigDB) using the Broad Institute Gene Set Enrichment Analysis (GSEA) software revealed similarities with many other gene expression signatures. In the case of MCF-7 cells, the most striking similarities were with IFN response gene signatures. These include signatures from the MSigDB Hallmark collection corresponding to Interferon alpha response, Interferon gamma response, and inflammatory response (Table 1). They also include numerous signatures associated with IFN responses from the MSigDB C2 curated gene set collection (Table 2). GSEA comparison of esiCDYL2-regulated genes and the MSigDB C2 collection further revealed strong similarities with gene expression signatures associated with the KEGG ‘antigen processing and presentation’ signature, suggesting CDYL2 inhibition might promote this central aspect of cancer cell immunogenicity (Table 2). In addition, CDYL2 RNAi induced gene expression signatures associated with the response to dsRNA (Table 2, ‘Geiss Response to dsRNA UP’ and ‘KEGG RIG-1-like receptor signaling pathway’). This suggests that, as was the case for loss or inhibition of DNMT1, HDACs, LSD1 and SETDB1 (Chiappinelli et al., (2015) Cell; Cuellar et al., (2017) Cell Biol; Sheng et al., (2018) Cell; Topper et al., (2019) Nat Rev Clin Oncol; Woods et al., (2015) Cancer Immunol Res), increased dsRNA levels might contribute to the observed IFN responses resulting from RNAi inhibition of CDYL2.

RNAi knock-down of CDYL2 followed by RNA-seq and GSEA analysis also revealed up-regulation of genes associated with IFN responses in MDA-MB-231 cells (Table 3, Table 5 and Table 4). A gene expression signature associated with the dsRNA response was also observed in this experiment (Table 5). Although the effect of CDYL2 RNAi on IFN response and dsRNA response gene expression signatures was less striking than in the case of MCF-7 cells, it was nonetheless statistically significant at the level of Nominal p-value. The reasons why CDYL2 RNAi more potently induced IFN and dsRNA response gene signatures in MCF-7 cells remain to be determined but could be related to the efficiency of RNAi knock-down, time of harvesting of RNA after transfection of cells with esiRNA, or simply differences in the intrinsic ability of each cell line to activate an IFN response.

Further evidence supporting a role for CDYL2 in regulating tumor cell immunogenicity came from RNA-seq analysis of the effects of stable over-expression of CDYL2 on MCF-7 cells. GSEA analysis of the resulting data-sets revealed that genes associated with IFN responses and antigen presentation were down-regulated in MCF-7 cells over-expressing CDYL2 compared to controls stably transfected with an empty vector (Table 7; note, the Enrichment Score (ES) and Normalized ES (NES) have negative values). Hence, while CDYL2 inhibition with RNAi induces IFN responses and related processes in MCF-7 cells, CDYL2 over-expression had the opposite effect. This further supports our hypothesis that CDYL2 levels negatively regulate the expression of IFN response and antigen processing and presentation genes, and that CDYL2 inhibition could be exploited therapeutically to induce expression of such genes in breast cancer.

Based on these gene expression changes, we propose that therapeutic inhibition of CDYL2 in breast cancer will induce an IFN response resulting in increased presentation of tumor antigens on MHC class 1 receptors and increased tumor cell attraction of T cells into the tumor microenvironment. While this could potentially be sufficient to increase T cell-directed elimination of cancer cells. This is expected to be especially the case in breast cancers expressing high levels of CDYL2 relative to normal breast tissue.

From a therapeutic point of view, we could envision the use of anti-CDYL2 antibodies—or modified versions thereof—to inhibit CDYL2 and induce an anti-tumour immune response in cancer patients. Towards this goal, we generated and tested an anti-CDYL2 antiserum. First, we cloned CDYL2 cDNA from MCF-7 cells into an N-terminal 6-Histidine tagging bacterial expression plasmid. Next we expressed this plasmid in DE3-pLysS E. coli, and purified CDYL2 using NiNTA agarose beads. The resulting purified protein was used to immunise two rabbits and generate polyclonal anti-CDYL2 sera (immunization conduced by Covalab, Lyon, France). The antisera thus produced were tested at various points along the immunisation protocol. One of the immunized rabbits produced an anti-CDYL2 antiserum that specifically reacted with a band of the expected molecular weight (FIG. 9A), whose expression was reduced by CDYL2 RNAi knock-down (FIG. 9B), and increased by HA-tagged CDYL2 over-expression in MCF-7 cells (FIG. 9C). Anti-HA western blotting confirmed that the anti-CDYL2 reactive band whose intensity increases after HA-CDYL2 over-expression has an identical molecular weight to HA-CDYL2 (FIG. 9C). By contrast, this antibody did not cross-react with transgenically expressed, HA-tagged CDYL/CDYL1, which anti-HA western blots revealed to run at a higher molecular weight than the anti-CDYL2 reactive band (FIG. 9D). We conclude that this antiserum specifically reacts with CDYL2.

### EXAMPLE 3: CDYL2 RNAI INHIBITS MDA-MB-231 LUNG TUMORIGENESIS IN VIVO

### Materials and Methods

### Preparation of Cells for the Assay

MDA-MB-231 cell lines stably transduced with either a negative control RNAi lentiviral vector (MISSION® pLKO.1-puro Non-Mammalian shRNA Control Plasmid DNA, SHC002, Sigma Aldrich) or a vector containing one of two distinct shRNA targeting CDYL2 (Sigma, shCDYL2 #1, TRCN0000359078; shCDYL2 #2, TRCN0000130741). The resulting transduced cells were then selected for 14 days using 2 μg/mL puromycin (Sigma, P8833) to eliminate cells that did not stably express the lentiviral vector. Knockdown of CDYL2 was validated by western blot using the antibody anti-CDYL2 (MyBiosource, MBS821304) according to standard protocols. CDYL2 knockdown was also validated by RT-PCR analysis using the primers (forward) ACCAACGGGGGATTGAACCTGC (table 8, SEQ ID NO:3) and (reverse) GGTGTCAGGGCATTGTTATCCGAGG (table 8, SEQ ID NO:4) in a Fast SYBR Green Master Mix (Applied Biosystems, #4385610) and an LC480 PCR machine (Roche). On the day of injection of cells into the mice, sub-confluent cell cultures were harvested by trypsinization and cells counted using a haemocytometer. Trypan blue staining of the cells at the time of counting confirmed the viability of the cells, as the vast majority excluded trypan blue dye. Cells were washed in ice-cold Phosphate Buffered saline (PBS), counted, and solutions of cells were prepared in cold PBS and kept on ice until the time of injection.

### Murine Tumorigenesis Assays

These assays were carried out in accordance with the protocols approved under the ethics committee application CECCAPP CLB-2020-002. CECCAPP is an ethics committee in animal experimentation in the Rhône-Alpes region, based in Lyon and registered with the Ministry of Higher Education and Research of France under number C2EA15.

The experiment was performed as follows: three groups of MDA-MB-231 cells described above (shControl, shCDYL2 #1, shCDYL2 #2) injected intravenously into the lateral tail vein of 8 weeks old nude (NMRI nu) mice and their ability to form tumors in the lungs was determined. Each mouse was injected with 2×10E+5 cells in 0.1 ml of PBS, and each group consisted of 7 mice. These analyses were carried out broadly according to previously published methods (Minn A J, et al. Nature. 2005; 436 (7050): 518-524; Kuperwasser et al., Cancer Research, 2005, Volume 65, Issue 14), except that we used CT imaging to detect lung tumor formation in living mice (as detailed below).

### Imaging Step

On the day of imaging, the anesthetized mouse (isoflurane) was slipped into the bed through which the gas anesthesia arrives. Thus the mouse remains motionless throughout the acquisition. Moreover, the receptacle also keeps the temperature of the mouse at 37° C. The testing period for the pulmonary observation is 2 minutes which induces an exposure of 746mGy (this dose is well less than the maximum tolerated dose of 2.6 Gy per exposure). The mouse was then returned to its cage. Acquisitions were made no more than once every two weeks.

### Results and Discussion:

These assays show that the shCDYL2-expressing MDA-MB-231 cells formed fewer and/or smaller lung tumours compared to the control cells. This was revealed visually in the CT scans (FIG. 10 A,B,C), and could be numerically extrapolated because lungs containing more and/or larger tumours have a reduced lung volume (Table 9).

In other words, CDYL2 inhibition by RNAi inhibited the ability of these cells to form tumours in the lungs after injection into the tail vein of mice. By day 56, two of the mice injected with shControl cells had to be euthanised for ethical reasons as they had too many tumours in their lungs. This is why this group has fewer mice than the two shCDYL2 treated groups.

The results of this assay are consistent with the idea that CDYL2 inhibition can reduce the tumorigenicity of breast cancer cells, in this case a triple-negative breast cancer cell line. They further support the attractiveness of CDYL2 as a therapeutic target in breast cancer.

### Table Section

TABLE 1
GSEA MSigDB Hallmarks enriched upon CDYL2 RNAi in MCF-7 cells compared to control RNAi. RNA-seq was used to compare the
relative expression of genes in MCF-7 cells treated with CDYL2 RNAi versus those treated with a control RNAi. The resulting
gene list was ranked from the most over-expressed in the CDYL2 RNAi dataset to the most down-regulated. This ranked gene
list was then compared to the GSEA MSigDB Hallmark gene set collection. Shown are the selected enriched gene expression
signatures ranked in order of their Normalised Enrichment Score (NES). This revealed enrichment of gene expression signatures
associated with the interferon response, as well as a number of other inflammation-associated gene signatures.

| | | | | NOM | FDR | FWER | RANK | LEADING |
|---|---|---|---|---|---|---|---|---|
| NAME | SIZE | ES | NES | p-val | q-val | p-val | AT MAX | EDGE |
| HALLMARK_INTERFERON_ALPHA_RESPONSE | 93 | 0.85202414 | 2.3963137 | 0 | 0 | 0 | 4551 | tags = 86%, |
| | | | | | | | | list = 11%, |
| | | | | | | | | signal = 97% |
| HALLMARK_INTERFERON_GAMMA_RESPONSE | 194 | 0.8040638 | 2.3467538 | 0 | 0 | 0 | 3906 | tags = 58%, |
| | | | | | | | | list = 10%, |
| | | | | | | | | signal = 64% |
| HALLMARK_TNFA_SIGNALING_VIA_NFKB | 196 | 0.71396554 | 2.082239 | 0 | 0 | 0 | 7748 | tags = 59%, |
| | | | | | | | | list = 19%, |
| | | | | | | | | signal = 72% |
| HALLMARK_ALLOGRAFT_REJECTION | 183 | 0.6662855 | 1.9243379 | 0 | 0 | 0 | 6411 | tags = 40%, |
| | | | | | | | | list = 16%, |
| | | | | | | | | signal = 47% |
| HALLMARK_INFLAMMATORY_RESPONSE | 190 | 0.6495075 | 1.8971618 | 0 | 0 | 0 | 5134 | tags = 37%, |
| | | | | | | | | list = 13%, |
| | | | | | | | | signal = 43% |
| HALLMARK_IL6_JAK_STAT3_SIGNALING | 85 | 0.68244517 | 1.893703 | 0 | 0 | 0 | 5944 | tags = 48%, |
| | | | | | | | | list = 15%, |
| | | | | | | | | signal = 56% |
| HALLMARK_COMPLEMENT | 190 | 0.6153743 | 1.7986497 | 0 | 8.89E−05 | 0.001 | 4897 | tags = 29%, |
| | | | | | | | | list = 12%, |
| | | | | | | | | signal = 33% |
| HALLMARK_APOPTOSIS | 157 | 0.594564 | 1.7259465 | 0 | 1.52E−04 | 0.002 | 5643 | tags = 25%, |
| | | | | | | | | list = 14%, |
| | | | | | | | | signal = 30% |
| HALLMARK_IL2_STAT5_SIGNALING | 190 | 0.5235451 | 1.527428 | 0 | 0.00647235 | 0.112 | 7565 | tags = 33%, |
| | | | | | | | | list = 19%, |
| | | | | | | | | signal = 41% |

TABLE 2
GSEA MSigDB C2 curated gene sets collection signatures that were enriched upon CDYL2 RNAi in MCF-7 cells compared
to control RNAi. RNA-seq was used to compare the relative expression of genes in MCF-7 cells treated with CDYL2
RNAi versus those treated with a control RNAi. The resulting gene list was ranked from the most over-expressed
in the CDYL2 RNAi dataset to the most down-regulated. This ranked gene list was then compared to the GSEA MSigDB
‘C2 curated gene sets’ collection. Shown are selected enriched gene expression signatures ranked in order
of their Normalised Enrichment Score (NES). This revealed enrichment of gene expression signatures associated
with the interferon alpha, beta and gamma responses, as well as an interferon signature in cancer.

| | | | | NOM | FDR | FWER | RANK | LEADING |
|---|---|---|---|---|---|---|---|---|
| NAME | SIZE | ES | NES | p-val | q-val | p-val | AT MAX | EDGE |
| BROWNE_INTERFERON_{—} | 65 | 0.92116886 | 2.5035763 | 0 | 0 | 0 | 2132 | tags = 83%, |
| RESPONSIVE_GENES | | | | | | | | list = 5%, |
| | | | | | | | | signal = 88% |
| SANA_RESPONSE_{—} | 74 | 0.85650414 | 2.357083 | 0 | 0 | 0 | 4433 | tags = 74%, |
| TO_IFNG_UP | | | | | | | | list = 11%, |
| | | | | | | | | signal = 83% |
| SANA_TNF_SIGNALING_UP | 76 | 0.8561752 | 2.3413148 | 0 | 0 | 0 | 1353 | tags = 53%, |
| | | | | | | | | list = 3%, |
| | | | | | | | | signal = 54% |
| MOSERLE_IFNA_RESPONSE | 31 | 0.9504861 | 2.3155174 | 0 | 0 | 0 | 1772 | tags = 97%, |
| | | | | | | | | list = 4%, |
| | | | | | | | | signal = 101% |
| HECKER_IFNB1_TARGETS | 87 | 0.8162935 | 2.2935858 | 0 | 0 | 0 | 2451 | tags = 54%, |
| | | | | | | | | list = 6%, |
| | | | | | | | | signal = 57% |
| DER_IFN_ALPHA_{—} | 73 | 0.8277265 | 2.269248 | 0 | 0 | 0 | 2663 | tags = 52%, |
| RESPONSE_UP | | | | | | | | list = 7%, |
| | | | | | | | | signal = 56% |
| RADAEVA_RESPONSE_{—} | 52 | 0.86436087 | 2.2681239 | 0 | 0 | 0 | 3702 | tags = 62%, |
| TO_IFNA1_UP | | | | | | | | list = 9%, |
| | | | | | | | | signal = 68% |
| BOSCO_INTERFERON_{—} | 72 | 0.82344323 | 2.2433825 | 0 | 0 | 0 | 3702 | tags = 56%, |
| INDUCED_ANTIVIRAL_MODULE | | | | | | | | list = 9%, |
| | | | | | | | | signal = 61% |
| DER_IFN_BETA_{—} | 101 | 0.790648 | 2.2273357 | 0 | 0 | 0 | 3237 | tags = 43%, |
| RESPONSE_UP | | | | | | | | list = 8%, |
| | | | | | | | | signal = 46% |
| REACTOME_INTERFERON_{—} | 56 | 0.8338998 | 2.202736 | 0 | 0 | 0 | 3060 | tags = 55%, |
| ALPHA_BETA_SIGNALING | | | | | | | | list = 8%, |
| | | | | | | | | signal = 60% |
| REACTOME_INTERFERON_{—} | 146 | 0.76679814 | 2.2004812 | 0 | 0 | 0 | 3906 | tags = 38%, |
| SIGNALING | | | | | | | | list = 10%, |
| | | | | | | | | signal = 42% |
| ZHANG_INTERFERON_RESPONSE | 23 | 0.93506724 | 2.1814709 | 0 | 0 | 0 | 2343 | tags = 91%, |
| | | | | | | | | list = 6%, |
| | | | | | | | | signal = 97% |
| GEISS_RESPONSE_{—} | 37 | 0.79821616 | 2.0357206 | 0 | 0 | 0 | 1891 | tags = 43%, |
| TO_DSRNA_UP | | | | | | | | list = 5%, |
| | | | | | | | | signal = 45% |
| KEGG_ANTIGEN_PROCESSING_{—} | 67 | 0.73790246 | 2.0054493 | 0 | 0 | 0 | 4421 | tags = 42%, |
| AND_PRESENTATION | | | | | | | | list = 11%, |
| | | | | | | | | signal = 47% |
| WORSCHECH_TUMOR_EVASION_{—} | 29 | 0.80273026 | 1.934113 | 0 | 1.32E−05 | 0.001 | 2308 | tags = 31%, |
| AND_TOLEROGENICITY_UP | | | | | | | | list = 6%, |
| | | | | | | | | signal = 33% |
| KEGG_RIG_I_LIKE_{—} | 62 | 0.6545148 | 1.7591536 | 0 | 0.00422434 | 0.484 | 5643 | tags = 32%, |
| RECEPTOR_SIGNALING_PATHWAY | | | | | | | | list = 14%, |
| | | | | | | | | signal = 37% |

TABLE 3
Enrichment of gene expression signatures associated with antigen presentation
and processing. Selected enrichment plots visualizing the indicated GSEA MSigDB
C2 curated gene sets collection signatures. These data relate to MCF-7 cells
treated with CDYL2 RNAi compared to control cells treated with a non-targeting
siRNA. Shown are profiles of the Running ES Score & Positions of GeneSet Members
on the Rank Ordered List. The associated statistics are shown in the table

| Dataset | preranked_MCF7_KD_alone |
|---|---|
| Phenotype | NoPhenotypeAvailable |
| Upregulated in class | na_pos |
| GeneSet | KEGG_ANTIGEN_PROCESSING_AND_PRESENTATION |
| Enrichment Score (ES) | 0.5307957 |
| Normalized Enrichment | 3.4366443 |
| Score (NES) | |
| Nominal p-value | 0.0 |
| FDR q-value | 1.530637E−5 |
| FWER p-Value | 0.003 |
| Dataset | preranked_MCF7_KD_alone |
| Phenotype | NoPhenotypeAvailable |
| Upregulated in class | na_pos |
| GeneSet | REACTOME_ANTIGEN_PROCESSING_CROSS_PRESENTATION |
| Enrichment Score (ES) | 0.43565646 |
| Normalized Enrichment | 2.7975485 |
| Score (NES) | |
| Nominal p-value | 0.0 |
| FDR q-value | 0.0029249415 |
| FWER p-Value | 0.794 |

TABLE 4
CDYL2 RNAi induces an interferon response gene signature in MDA-MB-231 cell line. Selected
GSEA MSigDB Hallmarks enriched upon CDYL2 RNAi in MDA-MB-231 cells compared to control RNAi.
These data relate to MDA-MB-231 cells treated with CDYL2 RNAi compared to control cells treated
with a non-targeting siRNA. Shown are profiles of the Running ES Score & Positions of GeneSet
Members on the Rank Ordered List. The associated statistics are shown in the table

| Dataset | MDAMB231_esiCD2_versus_MDAMB231_esiLuc |
|---|---|
| Phenotype | cdyl2_all_samples_cat.cls#MDAMB231_esiCD2_versus_MDAMB231_esiLuc_repos |
| Upregulated in class | MDAMB231_esiCD2 |
| GeneSet | HALLMARK_INTERFERON_ALPHA_RESPONSE |
| Enrichment Score (ES) | 0.504692 |
| Normalized Enrichment | 1.5324306 |
| Score (NES) | |
| Nominal p-value | 0.004470939 |
| FDR q-value | 0.08714676 |
| FWER p-Value | 0.103 |
| Dataset | MDAMB231_esiCD2_versus_MDAMB231_esiLuc |
| Phenotype | cdyl2_all_samples_cat.cls#MDAMB231_esiCD2_versus_MDAMB231_esiLuc_repos |
| Upregulated in class | MDAMB231_esiCD2 |
| GeneSet | HALLMARK_INTERFERON_GAMMA_RESPONSE |
| Enrichment Score (ES) | 0.44447333 |
| Normalized Enrichment | 1.472826 |
| Score (NES) | |
| Nominal p-value | 0.0013888889 |
| FDR q-value | 0.09225965 |
| FWER p-Value | 0.209 |

TABLE 5
GSEA MSigDB C2 curated gene sets collection signatures that were enriched upon CDYL2 RNAi in MDA-MB-231 cells compared to control
RNAi. RNA-seq was used to compare the relative expression of genes in MDA-MB-231 cells treated with CDYL2 RNAi versus those
treated with a control RNAi. The resulting gene list was ranked from the most over-expressed in the CDYL2 RNAi dataset to the
most down-regulated. This ranked gene list was then compared to the GSEA MSigDB ‘C2 curated gene sets’ collection. Shown
are selected enriched gene expression signatures ranked in order of their Normalised Enrichment Score (NES). This revealed
enrichment of gene expression signatures associated with the interferon signalling and tumor evasion and tolerogenicity.

| | | | | NOM | FDR | FWER p- | RANK | LEADING |
|---|---|---|---|---|---|---|---|---|
| NAME | SIZE | ES | NES | p-val | q-val | val | AT MAX | EDGE |
| ZHANGI_INTERFERON_{—} | 23 | 0.72339386 | 1.7277623 | 0.00168919 | 0.57765883 | 0.888 | 8162 | tags = 65%, |
| RESPONSE | | | | | | | | list = 20%, |
| | | | | | | | | signal = 82% |
| MOSERLE_IFNA_RESPONSE | 31 | 0.6615628 | 1.6825045 | 0 | 0.5698889 | 0.988 | 9337 | tags = 74%, |
| | | | | | | | | list = 23%, |
| | | | | | | | | signal = 97% |
| BROWNE_INTERFERON_{—} | 65 | 0.5806052 | 1.6543516 | 0 | 0.7417494 | 0.997 | 9103 | tags = 57%, |
| RESPONSIVE_GENES | | | | | | | | list = 23%, |
| | | | | | | | | signal = 73% |
| CHIANG_LIVER_CANCER_{—} | 24 | 0.6324977 | 1.5248917 | 0.03529412 | 0.98659104 | 1 | 8834 | tags = 54%, |
| SUBCLASS_INTERFERON_UP | | | | | | | | list = 22%, |
| | | | | | | | | signal = 69% |
| WORSCHECH_TUMOR_{—} | 29 | 0.6030707 | 1.5053093 | 0.03204047 | 0.9703892 | 1 | 6630 | tags = 41%, |
| EVASION_AND_{—} | | | | | | | | list = 16%, |
| TOLEROGENICITY_UP | | | | | | | | signal = 50% |
| GEISS_RESPONSE_{—} | 37 | 0.5679429 | 1.4921762 | 0.01597444 | 0.95655155 | 1 | 9506 | tags = 54%, |
| TO_DSRNA_UP | | | | | | | | list = 24%, |
| | | | | | | | | signal = 71% |
| EINAV_INTERFERON_{—} | 26 | 0.6105456 | 1.4813169 | 0.02684564 | 1 | 1 | 8162 | tags = 50%, |
| SIGNATURE_IN_CANCER | | | | | | | | list = 20%, |
| | | | | | | | | signal = 63% |
| BOSCO_INTERFERON_{—} | 72 | 0.50043344 | 1.4688625 | 0.01225115 | 1 | 1 | 9080 | tags = 47%, |
| INDUCED_ANTIVIRAL_MODULE | | | | | | | | list = 23%, |
| | | | | | | | | signal = 61% |

TABLE 6
Stable transgenic over-expression of CDYL2 represses expression of genes involved in the
interferon response in MCF-7 cell line. The indicated GSEA MSigDB Hallmarks negatively correlated
with CDYL2 over-expression in MCF-7 cells compared to control cells stably transfected with
an empty vector. Shown are profiles of the Running ES Score & Positions of GeneSet Members
on the Rank Ordered List. The associated statistics are shown in the table

| Dataset | MCF7_CDYL2_versus_MCF7_Vector |
|---|---|
| Phenotype | cdyl2_all_samples_cat.cls#MCF7_CDYL2_versus_MCF7_Vector_repos |
| Upregulated in class | MCF7_Vector |
| GeneSet | HALLMARK_INTERFERON_ALPHA_RESPONSE |
| Enrichment Score (ES) | −0.40990403 |
| Normalized Enrichment | −1.5868864 |
| Score (NES) | |
| Nominal p-value | 0.0 |
| FDR q-value | 0.01045875 |
| FWER p-Value | 0.028 |
| Dataset | MCF7_CDYL2_versus_MCF7_Vector |
| Phenotype | cdyl2_all_samples_cat.cls#MCF7_CDYL2_versus_MCF7_Vector_repos |
| Upregulated in class | MCF7_Vector |
| GeneSet | HALLMARK_INTERFERON_GAMMA_RESPONSE |
| Enrichment Score (ES) | −0.32045266 |
| Normalized Enrichment | −1.369899 |
| Score (NES) | |
| Nominal p-value | 0.0 |
| FDR q-value | 0.042379666 |
| FWER p-Value | 0.155 |

TABLE 7
GSEA MSigDB C2 curated gene sets collection signatures the expression of which was repressed in MCF-7 cells stably over-expressing
CDYL2 compared to control cells stably transfected with the empty vector. RNA-seq was used to compare the relative expression
of genes in MCF-7 cells over-expressing CDYL2 versus controls. The resulting gene list was ranked from the most over-expressed
in the CDYL2 RNAi dataset to the most down-regulated. This ranked gene list was then compared to the GSEA MSigDB ‘C2 curated
gene sets’ collection. This revealed an inverse correlation between CDYL2 over-expression and expression of gene expression
signatures associated with the interferon response, as well as antigen processing and cross-presentation. Nominal (NOM)
p-value, False Discovery Rate (FDR) q-value and FWER p-values indicated as zero are less than 0.001.

| | | | | NOM | FDR | FWER | RANK | LEADING |
|---|---|---|---|---|---|---|---|---|
| NAME | SIZE | ES | NES | p-val | q-val | p-val | AT MAX | EDGE |
| MOSERLE_IFNA_RESPONSE | 31 | −0.7358488 | −2.5625768 | 0 | 0 | 0 | 5560 | tags = 81%, |
| | | | | | | | | list = 20%, |
| | | | | | | | | signal = 101% |
| EINAV_INTERFERON_{—} | 25 | −0.7354067 | −2.3354008 | 0 | 2.84E−05 | 0.002 | 4471 | tags = 64%, |
| SIGNATURE_IN_CANCER | | | | | | | | list = 16%, |
| | | | | | | | | signal = 76% |
| REACTOME_CROSS_{—} | 45 | −0.6152415 | −2.3066409 | 0 | 5.26E−05 | 0.004 | 8487 | tags = 80%, |
| PRESENTATION_OF_{—} | | | | | | | | list = 31%, |
| SOLUBLE_EXOGENOUS_{—} | | | | | | | | signal = 115% |
| ANTIGENS_ENDOSOMES | | | | | | | | |
| RADAEVA_RESPONSE_{—} | 48 | −0.5988618 | −2.2925148 | 0 | 5.00E−05 | 0.004 | 5560 | tags = 52%, |
| TO_IFNA1_UP | | | | | | | | list = 20%, |
| | | | | | | | | signal = 65% |
| REACTOME_ANTIGEN_{—} | 70 | −0.5071787 | −2.0585754 | 0 | 0.00103037 | 0.124 | 9034 | tags = 69%, |
| PROCESSING_CROSS_{—} | | | | | | | | list = 32%, |
| PRESENTATION | | | | | | | | signal = 101% |
| BROWNE_INTERFERON_{—} | 64 | −0.5185457 | −2.03764 | 0 | 0.00134007 | 0.165 | 4050 | tags = 41%, |
| RESPONSIVE_GENES | | | | | | | | list = 15%, |
| | | | | | | | | signal = 47% |
| REACTOME_ANTIVIRAL_{—} | 65 | −0.473695 | −1.9114428 | 0.00367647 | 0.0046838 | 0.568 | 8410 | tags = 48%, |
| MECHANISM_BY_IFN_{—} | | | | | | | | list = 30%, |
| STIMULATED_GENES | | | | | | | | signal = 68% |
| ZHANG_INTERFERON_{—} | 23 | −0.5935327 | −1.8828089 | 0 | 0.00590645 | 0.672 | 4292 | tags = 65%, |
| RESPONSE | | | | | | | | list = 15%, |
| | | | | | | | | signal = 77% |

TABLE 8
Useful nucleotide and amino acid sequences for practicing the invention

| SEQ ID | |
|---|---|
| NO | Nucleotide or amino acid sequence |
| 1 (CDYL2 | MASGDLYEVERIVDKRKNKKGKWEYLIRWKGYGSTEDTWEPEHH |
| AA | LLHCEEFIDEFNGLHMSKDKRIKSGKQSSTSKLLRDSRGPSVEKLSH |
| sequence) | RPSDPGKSKGTSHKRKRINPPLAKPKKGYSGKPSSGGDRATKTVSY |
| | RTTPSGLQIMPLKKSQNGMENGDAGSEKDERHFGNGSHQPGLDLN |
| | DHVGEQDMGECDVNHATLAENGLGSALTNGGLNLHSPVKRKLEA |
| | EKDYVFDKRLRYSVRQNESNCRFRDIVVRKEEGFTHILLSSQTSDNN |
| | ALTPEIMKEVRRALCNAATDDSKLLLLSAVGSVFCSGLDYSYLIGRL |
| | SSDRRKESTRIAEAIRDFVKAFIQFKKPIVVAINGPALGLGASILPLCD |
| | IVWASEKAWFQTPYATIRLTPAGCSSYTFPQILGVALANEMLFCGR |
| | KLTAQEACSRGLVSQVFWPTTFSQEVMLRVKEMASCSAVVLEESK |
| | CLVRSFLKSVLEDVNEKECLMLKQLWSSSKGLDSLFSYLQDKIYEV |
| 2 (CDYL2 | atggcttctggggacctttacgaggttgaaaggattgtagacaagaggaagaacaagaaaggaaaatggg |
| nucleic | agtatcttatccgatggaaaggctacgggagcaccgaggacacgtgggagccggagcaccacctcttgca |
| acid | ctgtgaggagtttattgatgaattcaatgggttgcacatgtccaaggacaagaggatcaagtcagggaagca |
| sequence) | gtccagtacctccaagctgctgcgtgacagtcgaggcccgtcggttgagaaactgtcccacagaccttcag |
| | atcctggaaagagcaaggggacctcccataaacggaagcgaattaaccctcccctggccaagccaaaaa |
| | aagggtattcaggcaagccctcttcaggaggtgacagggccaccaagacggtgtcttacaggactacccc |
| | cagtggtttgcaaataatgcccctgaaaaagtctcagaacgggatggaaaatggggacgccggctctgag |
| | aaggatgagaggcactttggaaatgggtcccatcagcctggcttggatttgaatgatcatgttggagagcaa |
| | gatatgggtgaatgtgacgtgaatcacgctacactggcggagaacgggctcggctctgctctgaccaacg |
| | ggggattgaacctgcacagtccagtgaagaggaagctggaagcggagaaggactacgtctttgacaaaa |
| | ggctcagatacagtgtccgccagaatgaaagcaactgtcggtttcgagacatcgttgtgcggaaggaagaa |
| | gggttcacgcacatcctgctgtccagtcagacctcggataacaatgccctgacacctgagatcatgaaaga |
| | agtccggcgagcgctctgcaacgcagccacagacgacagcaaactgctgctcctcagcgcagtgggga |
| | gcgtgttctgcagcggcctggattattcctacctaattggccggttgtccagcgaccggcgaaaggagagc |
| | actcggattgcagaagccatcagggactttgtgaaggcctttatccagtttaagaagcctatcgtggtggcca |
| | tcaatgggccggccctgggcctgggtgcctccatcctgcccctctgtgacatcgtgtgggccagtgagaag |
| | gcctggttccagacgccctacgccaccatccgcctcacgcctgctggctgctcctcctacaccttcccccag |
| | atcctgggcgtcgcgctggccaatgagatgctgttctgtgggcggaagctcaccgcccaggaggcctgca |
| | gcagggggctggtgtcgcaggtcttctggcccaccacgttcagccaggaggtcatgctgcgggtcaagga |
| | gatggcatcctgcagtgccgtggtgttagaggagtccaaatgcctcgtgcggagcttcctgaaatcagtgct |
| | ggaagacgtgaacgagaaggaatgcctcatgctcaagcagctctggagctcctccaaaggccttgactcc |
| | cttttcagctacctgcaggacaaaatttatgaagtctga |
| 3 (CDYL2 | accaacgggggattgaacctgc |
| forward | |
| PCR | |
| primer) | |
| 4 (CDYL2 | ggtgtcagggcattgttatccgagg |
| PCR | |
| reverse | |
| primer) | |

TABLE 9
Quantification of mean lung volume (mm3) by CT scan 56
days after injection of shControl, shCDYL2 #1 or shCDYL2
#2 MDA-MB-231 cells into the tail vein of nude mice.

| shRNA plasmid | Mean Lung Vol (day 56) | Mann-Whitney p-val |
|---|---|---|
| shControl (n = 5) | 429,615 mm3 | |
| shCDYL2 #1 (n = 7) | 512,380 mm3 | <0.05 |
| shCDYL2 #2 (n = 7) | 596,88 mm3 | <0.05 |

### REFERENCES

Throughout this application, various references describe the state of the art to which this invention pertains. The disclosures of these references are hereby incorporated by reference into the present disclosure.
1. Dawson, M. A. & Kouzarides, T. Cancer epigenetics: from mechanism to therapy. Cell 150, 12-27 (2012).
2. Tam, W. L. & Weinberg, R. A. The epigenetics of epithelial-mesenchymal plasticity in cancer. Nat Med 19, 1438-49 (2013).
3. Banerjee, K. & Resat, H. Constitutive activation of STAT3 in breast cancer cells: A review. International Journal of Cancer 138, 2570-2578 (2016).
4. Marotta, L. L. et al. The JAK2/STAT3 signaling pathway is required for growth of CD44+CD24-stem cell-like breast cancer cells in human tumors. The Journal of clinical investigation 121, 2723-2735 (2011).
5. Shostak, K. & Chariot, A. NF-kappaB, stem cells and breast cancer: the links get stronger. Breast cancer research: BCR 13, 214 (2011).
6. Xia, Y., Shen, S. & Verma, I. M. NF-kappaB, an active player in human cancers. Cancer Immunol Res 2, 823-30 (2014).
7. Yang, L., Han, S. & Sun, Y. An IL6-STAT3 loop mediates resistance to PI3K inhibitors by inducing epithelial-mesenchymal transition and cancer stem cell expansion in human breast cancer cells. Biochem. Biophys. Res. Commun. 453, 582-587 (2014).
8. Zhou, J. et al. NF-kappaB pathway inhibitors preferentially inhibit breast cancer stem-like cells. Breast Cancer Res Treat 111, 419-27 (2008).
9. Chang, C.-J. et al. EZH2 promotes expansion of breast tumor initiating cells through activation of RAF1-β-catenin signaling. Cancer Cell 19, 86-100 (2011).
10. Curry, E. et al. Dual EZH2 and EHMT2 histone methyltransferase inhibition increases biological efficacy in breast cancer cells. Clin Epigenetics 7, 84 (2015).
11. Dong, C. et al. G9a interacts with Snail and is critical for Snail-mediated E-cadherin repression in human breast cancer. J Clin Invest 122, 1469-86 (2012).
12. Kleer, C. G. et al. EZH2 is a marker of aggressive breast cancer and promotes neoplastic transformation of breast epithelial cells. Proceedings of the National Academy of Sciences 100, 11606-11611 (2003).
13. Ji, H., Sang, M., Liu, F., Ai, N. & Geng, C. miR-124 regulates EMT based on ZEB2 target to inhibit invasion and metastasis in triple-negative breast cancer. Pathol. Res. Pract. 215, 697-704 (2019).
14. Lv, X.-B. et al. miR-124 suppresses multiple steps of breast cancer metastasis by targeting a cohort of pro-metastatic genes in vitro. Chinese journal of cancer 30, 821 (2011).
15. Wang, X. et al. Sulforaphane improves chemotherapy efficacy by targeting cancer stem cell-like properties via the miR-124/IL-6R/STAT3 axis. Sci Rep 6, 36796 (2016).
16. Cao, J. et al. Identification of microRNA-124 in regulation of Hepatocellular carcinoma through BIRC3 and the NF-κB pathway. Journal of Cancer 9, 3006 (2018).
17. Hatziapostolou, M. et al. An HNF4α-miRNA Inflammatory Feedback Circuit Regulates Hepatocellular Oncogenesis. Cell 147, 1233-1247 (2011).
18. Mehta, A. K. et al. Regulation of autophagy, NF-κB signaling, and cell viability by miR-124 in KRAS mutant mesenchymal-like NSCLC cells. Sci. Signal. 10, eaam6291 (2017).
19. Olarerin-George, A. O., Anton, L., Hwang, Y.-C., Elovitz, M. A. & Hogenesch, J. B. A functional genomics screen for microRNA regulators of NF-kappaB signaling. BMC Biology 11, 19 (2013).
20. Wang, Y. et al. miR-124-3p functions as a tumor suppressor in breast cancer by targeting CBL. BMC Cancer 16, 826 (2016).
21. Zhou, H. et al. Regulatory Network of Two Tumor-Suppressive Noncoding RNAs Interferes with the Growth and Metastasis of Renal Cell Carcinoma. Molecular Therapy-Nucleic Acids 16, 554-565 (2019).
22. Dorus, S., Gilbert, S. L., Forster, M. L., Barndt, R. J. & Lahn, B. T. The CDY-related gene family: coordinated evolution in copy number, expression profile and protein sequence. Hum Mol Genet 12, 1643-50 (2003).
23. Fischle, W., Franz, H., Jacobs, S. A., Allis, C. D. & Khorasanizadeh, S. Specificity of the chromodomain Y chromosome family of chromodomains for lysine-methylated ARK(S/T) motifs. The Journal of biological chemistry 283, 19626-35 (2008).
24. Franz, H., Mosch, K., Soeroes, S., Urlaub, H. & Fischle, W. Multimerization and H3K9me3 binding are required for CDYL1b heterochromatin association. The Journal of biological chemistry 284, 35049-59 (2009).
25. Mulligan, P. et al. CDYL Bridges REST and Histone Methyltransferases for Gene Repression and Suppression of Cellular Transformation. Mol Cell 32, 718-26 (2008).
26. Wu, H. et al. Short-Form CDYLb but not long-form CDYLa functions cooperatively with histone methyltransferase G9a in hepatocellular carcinomas. Genes Chromosomes Cancer 52, 644-55 (2013).
27. Zhang, Y. et al. Corepressor protein CDYL functions as a molecular bridge between polycomb repressor complex 2 and repressive chromatin mark trimethylated histone lysine 27. The Journal of biological chemistry 286, 42414-25 (2011).
28. Michailidou, K. et al. Large-scale genotyping identifies 41 new loci associated with breast cancer risk. Nature genetics 45, 353-61, 361e1-2 (2013).
29. Mulligan, P. et al. A SIRT1-LSD1 Corepressor Complex Regulates Notch Target Gene Expression and Development. Mol Cell 42, 689-99 (2011).
30. Dobin, A. et al. STAR: ultrafast universal RNA-seq aligner. Bioinformatics 29, 15-21 (2013).
31. Langmead, B. & Salzberg, S. L. Fast gapped-read alignment with Bowtie 2. Nat. Methods 9, 357-359 (2012).
32. Zhang, Y. et al. Model-based analysis of ChIP-Seq (MACS). Genome Biol. 9, R137 (2008).
33. Huang, W., Loganantharaj, R., Schroeder, B., Fargo, D. & Li, L. PAVIS: a tool for Peak Annotation and Visualization. Bioinformatics 29, 3097-3099 (2013).
34. Cancer Genome Atlas Network. Comprehensive molecular portraits of human breast tumours. Nature 490, 61-70 (2012).
35. Chen, D.-T. et al. Proliferative genes dominate malignancy-risk gene signature in histologically-normal breast tissue. Breast Cancer Res. Treat. 119, 335-346 (2010).
36. Kretschmer, C. et al. Identification of early molecular markers for breast cancer. Mol. Cancer 10, 15 (2011).
37. Ellis, M. J. et al. Connecting genomic alterations to cancer biology with proteomics: the NCI Clinical Proteomic Tumor Analysis Consortium. Cancer Discov 3, 1108-1112 (2013).
38. Alam, M., Rajabi, H., Ahmad, R., Jin, C. & Kufe, D. Targeting the MUC1-C oncoprotein inhibits self-renewal capacity of breast cancer cells. Oncotarget 5, 2622-34 (2014).
39. Bakiri, L. et al. Fra-1/AP-1 induces EMT in mammary epithelial cells by modulating Zeb1/2 and TGFbeta expression. Cell Death Differ 22, 336-50 (2015).
40. Jiao, X. et al. c-Jun induces mammary epithelial cellular invasion and breast cancer stem cell expansion. The Journal of biological chemistry 285, 8218-26 (2010).
41. Kufe, D. W. MUC1-C oncoprotein as a target in breast cancer: activation of signaling pathways and therapeutic approaches. Oncogene 32, 1073-81 (2013).
42. Nair, R. et al. c-Myc and Her2 cooperate to drive a stem-like phenotype with poor prognosis in breast cancer. Oncogene 33, 3992-4002 (2014).
43. Piva, M. et al. Sox2 promotes tamoxifen resistance in breast cancer cells. EMBO Mol Med 6, 66-79 (2014).
44. Yu, F. et al. Kruppel-like factor 4 (KLF4) is required for maintenance of breast cancer stem cells and for cell migration and invasion. Oncogene 30, 2161-72 (2011).
45. Rhodes, D. R. et al. ONCOMINE: A Cancer Microarray Database and Integrated Data-Mining Platform. Neoplasia 6, 1-6 (2004).
46. Shimo, T. et al. Pathogenic role of connective tissue growth factor (CTGF/CCN2) in osteolytic metastasis of breast cancer. J Bone Miner Res 21, 1045-59 (2006).
47. Singh, J. K., Simoes, B. M., Howell, S. J., Farnie, G. & Clarke, R. B. Recent advances reveal IL-8 signaling as a potential key to targeting breast cancer stem cells. Breast cancer research: BCR 15, 210 (2013).
48. Sullivan, N. J. et al. Interleukin-6 induces an epithelial-mesenchymal transition phenotype in human breast cancer cells. Oncogene 28, 2940-7 (2009).
49. Gatti, V. et al. p63 at the Crossroads between Stemness and Metastasis in Breast Cancer. International Journal of Molecular Sciences 20, 2683 (2019).
50. Romagnoli, M. et al. Epithelial-to-mesenchymal transition induced by TGF-beta1 is mediated by Blimp-1-dependent repression of BMP-5. Cancer research 72, 6268-78 (2012).
51. Dhasarathy, A., Kajita, M. & Wade, P. A. The Transcription Factor Snail Mediates Epithelial to Mesenchymal Transitions by Repression of Estrogen Receptor-α. Mol Endocrinol 21, 2907-2918 (2007).
52. Lin, Y.-C., Lee, Y.-C., Li, L.-H., Cheng, C.-J. & Yang, R.-B. Tumor suppressor SCUBE2 inhibits breast-cancer cell migration and invasion through the reversal of epithelial-mesenchymal transition. J. Cell. Sci. 127, 85-100 (2014).
53. Yin, L., Castagnino, P. & Assoian, R. K. ABCG2 expression and side population abundance regulated by a transforming growth factor beta-directed epithelial-mesenchymal transition. Cancer Res. 68, 800-807 (2008).
54. Mozzetta, C. et al. The Histone H3 Lysine 9 Methyltransferases G9a and GLP Regulate Polycomb Repressive Complex 2-Mediated Gene Silencing. Molecular Cell 53, 277-289 (2014).
55. Conaco, C., Otto, S., Han, J.-J. & Mandel, G. Reciprocal actions of REST and a microRNA promote neuronal identity. PNAS 103, 2422-2427 (2006).
56. Huber, M. A. et al. NF-kappaB is essential for epithelial-mesenchymal transition and metastasis in a model of breast cancer progression. J Clin Invest 114, 569-81 (2004).
57. Puisieux, A., Brabletz, T. & Caramel, J. Oncogenic roles of EMT-inducing transcription factors. Nat Cell Biol 16, 488-94 (2014).
58. Sarrio, D. et al. Epithelial-mesenchymal transition in breast cancer relates to the basal-like phenotype. Cancer research 68, 989-97 (2008).
59. Shibue, T. & Weinberg, R. A. EMT, CSCs, and drug resistance: the mechanistic link and clinical implications. Nat Rev Clin Oncol 14, 611-629 (2017).

1. A method of preventing or treating cancer in a patient in need thereof, comprising
administering to the patient a therapeutically effective amount of a CDYL2 antagonist.

2. The method according to claim 1 wherein said cancer is a drug resistant cancer.

3. The method according to of claim 1, wherein said CDYL2 antagonist directly binds to CDYL2 protein or a nucleic sequence encoding the CRYL2 protein and promotes the expression of genes that regulate an anti-tumor immune response.

4. The method according to of claim 1 wherein said CDYL2 antagonist is selected from the group consisting of a small organic molecule; an inhibitor of CDYL2 gene expression; an anti-CDYL2 neutralizing antibody; and an anti-CDYL2 aptamer.

5. The method according to claim 4 wherein the inhibitor of CDYL2 gene expression is selected from the group consisting of an antisense oligonucleotide, a nuclease, siRNA, shRNA and a ribozyme nucleic acid sequence.

6. The method according to claim 1 wherein said cancer is a solid tumor or lymphoma/leukemia.

7. The method according to claim 6 wherein the solid tumor is selected from the group consisting of breast cancer, colorectal cancer, lung cancer, oesophagus cancer and renal cancer.

8. A method to activate the anti-tumoral immune response of a patient affected with a cancer, comprising
administering to the patient a therapeutically effective amount of a CDYL2 antagonist.

9. The method according to claim 8 wherein said cancer is a drug resistant cancer.

10. The method according to claim 8, wherein said CDYL2 antagonist binds directly to CDYL2 protein or a nucleic acid sequence encoding the CDYL2 protein and promotes the expression of genes that regulate an anti-tumor immune response.

11. The method according to claim 8, wherein said CDYL2 antagonist is selected from the group consisting of a small organic molecule; an inhibitor of CDYL2 gene expression; an anti-CDYL2 neutralizing antibody; and an anti-CDYL2 aptamer.

12. The method according to claim 11 wherein the inhibitor of CDYL2 gene expression is selected from the group consisting of an antisense oligonucleotide, a nuclease, siRNA, shRNA and a ribozyme nucleic acid sequence.

13. The method according to claim 8 wherein said cancer is a solid tumor or lymphoma/leukaemia.

14. The method according to claim 13, wherein the solid tumor is selected from the group consisting of breast cancer, colorectal cancer, lung cancer, oesophagus cancers and renal cancer.

15. The method of claim 3, wherein the nucleic acid sequence is DNA or mRNA.

16. The method of claim 10, wherein the nucleic acid sequence is DNA or mRNA.
